SEQUENCE LISTING (16 sequences)

SEQ ID NO: 1 - protein, 10 aa, Artificial Sequence
  CDR1 of the light chain of m906
QSLLHSNGYN

SEQ ID NO: 2 - protein, 3 aa, Artificial Sequence
  CDR2 of the light chain of m906
YLG

SEQ ID NO: 3 - protein, 10 aa, Artificial Sequence
  CDR3 of the light chain of m906
CMQSLQTPWT

SEQ ID NO: 4 - protein, 11 aa, Artificial Sequence
  CDR1 of the heavy chain of m906
GGTFTGYYMHW

SEQ ID NO: 5 - protein, 9 aa, Artificial Sequence
  CDR2 of the heavy chain of m906
NSGGTNYAQ

SEQ ID NO: 6 - protein, 15 aa, Artificial Sequence
  CDR3 of the heavy chain of m906
LSSGYSGYFDYWGQG

SEQ ID NO: 7 - protein, 219 aa, Artificial Sequence
  Light chain of m906
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 451 aa, Artificial Sequence
  Heavy chain of m906
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 59 bp, Artificial Sequence
  RNAi TRCN0000373085 Forward
tcccagcgttggagagtccaaattctcgagaatttggactctccaacgcttttttcggg

SEQ ID NO: 10 - DNA, 51 bp, Artificial Sequence
  RNAi TRCN0000373085 Reverse
aaaaaagcgttggagagtccaaattctcgagaatttggactctccaacgct

SEQ ID NO: 11 - DNA, 55 bp, Artificial Sequence
  RNAi TRCN0000373034 Forward
tccccgttccctgaaaccgttaaactcgagtttaacggtttcagggaacgttttt

SEQ ID NO: 12 - DNA, 55 bp, Artificial Sequence
  RNAi TRCN0000373034 Reverse
cgggaaaaacgttccctgaaaccgttaaactcgagtttaacggtttcagggaacg

SEQ ID NO: 13 - DNA, 55 bp, Artificial Sequence
  RNAi TRCN0000073460 Forward
tccccatgtaccttgaagtgcaatctcgagattgcacttcaaggtacatgttttt

SEQ ID NO: 14 - DNA, 55 bp, Artificial Sequence
  RNAi TRCN0000073460 Reverse
cgggaaaaacatgtaccttgaagtgcaatctcgagattgcacttcaaggtacatg

SEQ ID NO: 15 - protein, 113 aa, Artificial Sequence
  Variable domain of the light chain of m906
DVVMTQSPLSLPVTPGEPASISCRSSQSLLHSNGYNFLDWYLQKPGQSPQLLIYLGSNRASGVPDRFSGSGSGTDFTLKISRVEADDVGVYYCMQSLQTPWTFGHGTKVEIKR

SEQ ID NO: 16 - protein, 120 aa, Artificial Sequence
  Variable domain of the heavy chain of m906
EVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVS

(19) United States
(12) Patent Application Publication
(10) Pub. No.: US 2023/0144142 A1
(43) Pub. Date: May 11, 2023

(54) ANTI-CD56 ANTIBODY-DRUG CONJUGATES AND THEIR USE IN THERAPY

(71) Applicant: MCSAF INSIDE ONCOLOGY, TOURS (FR)

(72) Inventors: Ludovic JUEN, TOURS (FR); Christine BALTUS, LA RICHE (FR); Audrey DESGRANGES, VEIGNÉ (FR); Antoine TOUZÉ, MONTBAZON (FR); Thibault KERVARREC, VEIGNÉ (FR); Valérie LEBLOND, FONDETTES (FR); Mahtab SAMIMI, SAINT-CYR-SUR-LOIRE (FR)

(21) Appl. No.: 17/802,274

(22) PCT Filed: Feb. 26, 2021
(86) PCT No.: PCT/FR2021/050332

§ 371 Date: Aug. 25, 2022

(30) Foreign Application Priority Data
Feb. 27, 2020 (FR) FR2001974

(51) Int. Cl. A61K 47/68; A61K 31/40; A61P 35/00

(52) U.S. Cl. CPC A61K 47/6849; A61K 31/40; A61P 35/00

(57) ABSTRACT

The invention relates to antibody-drug conjugates and to their use in therapy, in particular for treating CD56+ cancers.

### TECHNICAL FIELD

The present invention relates to novel antibody-drug conjugates comprising an antibody directed against the CD56 antigen coupled to a cytotoxic drug and to theft use as a drug, in particular in anti-cancer therapy.

### PRIOR ART

An Antibody-Drug Conjugate (“ADC”) constitutes a means for the selective delivery of a cytotoxic drug. Thus, the antibody-drug conjugate can be used to combine the specificity of targeting by antibodies with powerful novel effector functions of the agents with which they are conjugated.

The general structure of an antibody-drug conjugate is that of formula (I). The portion linking the antibody and the drug is termed the linker. It can be grafted onto the antibody via at least one of eight cysteines forming the 4 interchain disulfide bridges. The number of molecules of cytotoxic drugs grafted onto the antibody determines a ratio known as the Drug-to-Antibody Ratio (DAR).

After fixation to its target antigen, the antibody is internalized in the cell by endocytosis mediated by receptors. The vesicles fuse with lysosomes where the cytotoxic drug is released from the antibody via different mechanisms. The active cytotoxic drug then acts on the cell, inducing its death, and sometimes on the adjacent cancer cells by transport or diffusion into the environment. Thus, the antibody is principally used as a vector and delivers the cytotoxic drug to the target cell.

Merkel cell carcinoma (MCC) is an aggressive skin cancer which occurs in elderly subjects. Until recently, the treatment of inoperable metastatic patients employed polychemotherapy based on platinum salts, without survival benefit. The use of an immunotherapy targeting PD-L1 (avelumab) as a first line therapy has been able to obtain an objective response in approximately 50% of patients with inoperable CCM with a durable response in half of the responding patients. However, the persistence of patients who have not responded has prompted the development of novel therapeutic strategies. In this context, the development of a targeted therapy in CCM appears to constitute a promising strategy.

In this context, CD56 has been identified as a therapeutic target that is strongly expressed by the majority of CCMs. In fact, IMGN901, which is an anti-CD56 ADC coupled to mertansine (DM1) by a first-generation technology, inhibits the polymerization of tubulin. An acceptable tolerance of IMGN901 was able to be demonstrated by several phase I studies in patients with solid tumors expressing CD56, including cases of CCM. In addition, a phase II study has been proposed with the same molecule in small cell lung carcinoma, in combination with chemotherapy (cisplatin-etoposide). That study did not demonstrate any survival benefit because of the frequent occurrence of secondary effects in the group treated with IMGN901 and in particular febrile neutropenia (infection linked to a reduction in neutrophiles), suggesting a non specific toxicity of the substance. Thus, there is a need for the development of cytotoxic anti-CD56-drug conjugates that are more homogeneous, more stable and therefore more effective, and/or that generate fewer secondary effects. In addition, the technology described in the present patent can be used to deliver the cytotoxic molecule specifically to tumor cells while limiting its non-specific release into the circulation, thereby limiting the non-specific toxicities induced by the drug.

### SUMMARY OF THE INVENTION

A first objective of the invention concerns an antibody-drug conjugate with the following formula (I):

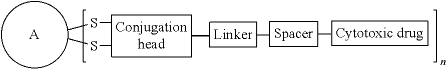

in which:
A is an anti-CD56 antibody or an antibody fragment;

the attachment head is represented by one of the following formulae:

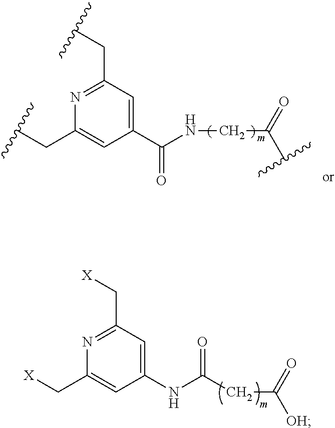

the linker is a cleavable linker selected from the following formulae:

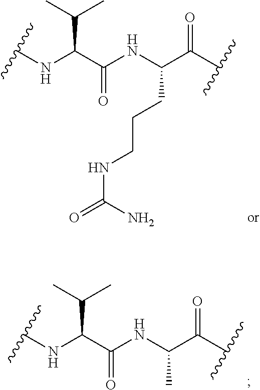 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the following formula:

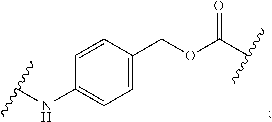 [SMILES: *Nc1ccc(COC(*)=O)cc1]

m is an integer from 1 to 10;
n is an integer from 1 to 4.

In a second objective, the invention concerns a composition comprising one or more antibody-drug conjugate(s) in accordance with the invention.

In a third objective, the invention concerns an antibody-drug conjugate in accordance with the invention or a composition in accordance with the invention, for use as a drug.

In a fourth objective, the invention concerns an antibody-drug conjugate in accordance with the invention or a composition in accordance with the invention, for use in the treatment of a CD56+ cancer.

In a fifth objective, the invention concerns a method for the preparation of an antibody-drug conjugate in accordance with the invention, comprising the following steps:

(i) preparing a cytotoxic conjugate by coupling a attachment head with formula:

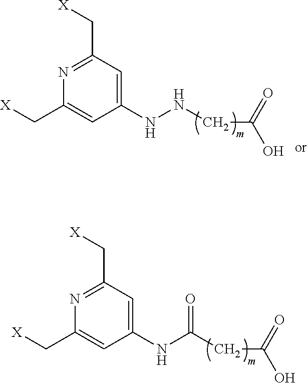

to a compound with formula:

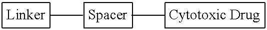

in which:
the linker is a cleavable linker selected from the following formulae:

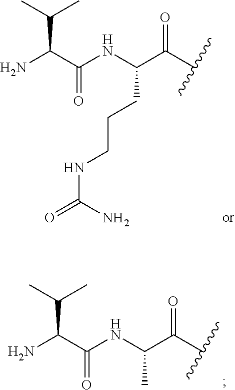 [SMILES: *C(=O)[C@H](CCCNC(N)=O)NC(=O)[C@@H](N)C(C)C ; *C(=O)[C@H](C)NC(=O)[C@@H](N)C(C)C]

the spacer is represented by the following formula:

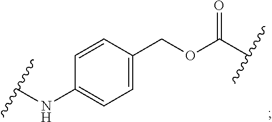 [SMILES: *Nc1ccc(COC(*)=O)cc1]

### X is Br, Cl, I or F;

m is an integer from 1 to 10, advantageously from 2 to 7, from 3 to 6, advantageously equal to 4 or 5; and
(ii) reacting the cytotoxic conjugate obtained in step (i) with an anti-CD56 antibody or an anti-CD56 antibody fragment.

Preferably, the method for the preparation of an antibody-drug conjugate in accordance with the invention comprises a step that consists of reacting MMAE 6-(2,6-bis(bromomethyl)pyridin-4-yl)amido-N-hexanamide-valine-citrulline-p-aminobenzoyl carbamate or MMAE 6-((2,6-bis(bromomethyl)pyridin-4-yl)amino)-6-oxohexanamide-valine-citrulline-p-aminobenzoyl carbamate with an anti-CD56 antibody or an anti-CD56 antibody fragment.

### DETAILED DESCRIPTION

### Definitions

The term “cytotoxic conjugate” designates a conjugate that comprises a cytotoxic drug. In the context of the present invention, a cytotoxic conjugate designates a conjugate with the following formula (II):

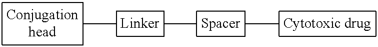

in which:
the attachment head is represented by one of the following formulae:

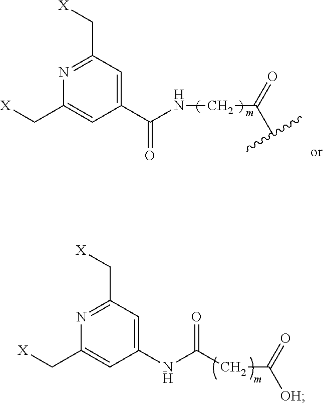

the linker is a cleavable linker selected from the following formulae:

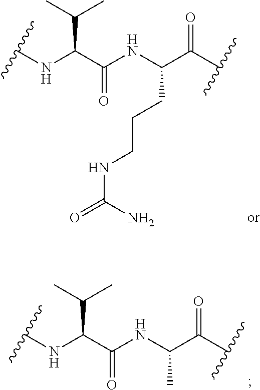 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the following formula:

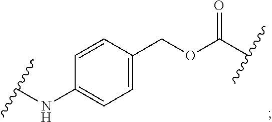 [SMILES: *Nc1ccc(COC(*)=O)cc1]

### X is Br, Cl, I or F;

m is an integer from 1 to 10.

The term “cytotoxic drug” designates any natural or synthesized molecule that is capable of inhibiting or preventing cell function and/or development. The term “cytotoxic” means the property of a chemical or biological agent of altering cells, possibly up to their destruction.

In a particular embodiment of the invention, the cytotoxic drug is selected from any compound that has obtained a MA and that is used in anti-cancer or anti-inflammatory therapy, and any molecule undergoing clinical evaluation for anti-cancer or anti-inflammatory therapy. The cytotoxic drug will be selected, for example, from paclitaxel (Taxol®) or docetaxel (Taxotére®) or one of its derivatives, topotecan, bortezomib, daunorubicin, analogs of daunorubicin, vincristine, mitomycin C, retinoic acid, methotrexate, Ilomedine®, aspirin, an IMID (Immunomodulatory imide drug), lenalidomide, pomalidomide.

In another particular embodiment of the invention, the cytotoxic drug is selected from the group constituted by duocarmycin and its analogs, dolastatins, combretastatin and its analogs, calicheamicin, N-acetyl-y-calicheamicin (CMC), a derivative of calicheamicin, maytansine and its analogs such as a derivative of the maytansinoid type, for example DM1 and DM4, auristatins and their derivatives, such as auristatin E, auristatin EB (AEB), auristatin EFP (AEFP), monomethyl auristatin E (MMAE), monomethyl auristatin F (MMAF), tubulysin, disorazole, epothilones, echinomycin, estramustine, cemadotin, eleutherobin, methopterin, actinomycine, mitomycin A, camptothecin, a derivative of camptothecin, SN38, TK1, amanitin, a pyrrolobenzodiazepine, a dimer of pyrrolobenzodiazepine, a pyrrolopyridodiazepine, a dimer of pyrrolopyridodiazepine, a DNA intercalating agent, an inhibitor of histone deacetylase, or an inhibitor of tyrosine kinase. In another particular embodiment of the invention, the drug M is selected from pseudomonas exotoxin (PE), deBouganin, Bouganin, the diphtheria toxin (DT) and ricin.

In a particular embodiment, the cytotoxic drug is selected from methotrexate, IMID, duocarmycin, combretastatin, calicheamicin, monomethyl auristatin E (MMAE), monomethyl auristatin F (MMAF), maytansine, DM1, DM4, SN38, amanitin, pyrrolobenzodiazepine, pyrrolobenzodiazepine dimer, pyrrolopyridodiazepine, pyrrolopyridodiazepine dimer, an inhibitor of histone deacetylase, an inhibitor of tyrosine kinase, and ricin, preferably MMAE, represented by the following formula:

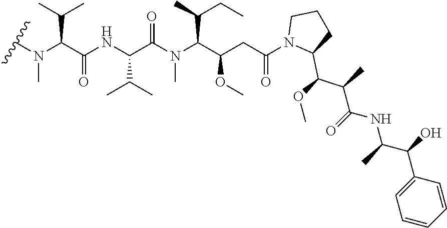 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

Thus, in a particularly preferred embodiment of the invention, the cytotoxic conjugate has the following formula (IIa):

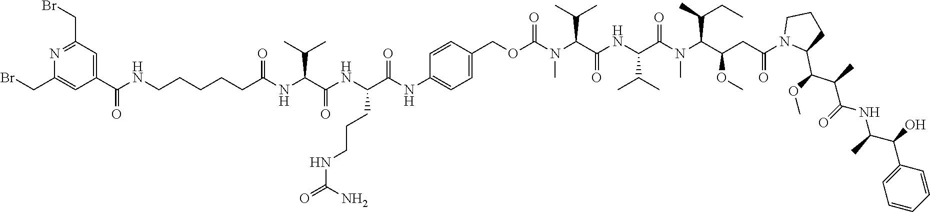 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCNC(=O)c2cc(CBr)nc(CBr)c2)C(C)C)cc1)C(C)C]

or the following formula (Ila′):

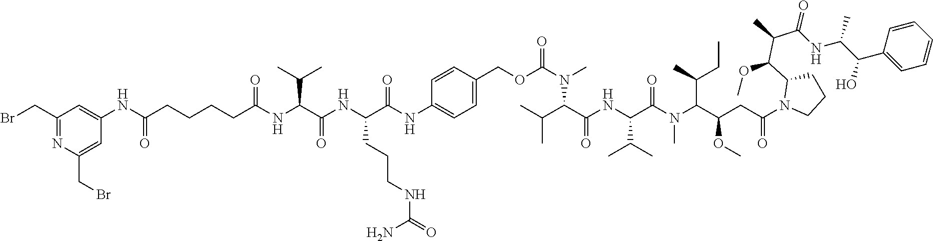 [SMILES: CC[C@H](C)C([C@@H](CC(=O)N1CCC[C@H]1[C@@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCC(=O)Nc2cc(CBr)nc(CBr)c2)C(C)C)cc1)C(C)C]

The term “antibody”, also known as “immunoglobulin,” designates a heterotetramer constituted by two heavy chains each of approximately 50-70 kDa (termed the H chains, for Heavy) and two light chains each of approximately 25 kDa (termed the L chains, for Light), linked together by intracatenary and intercatenary disulfide bridges. Each chain is constituted, at the N-terminal position, by a variable region or domain known as VL for the light chain, VH for the heavy chain and, at the C-terminal position, by a constant region constituted by a single domain termed CL for the light chain and three or four domains termed CH1, CH2, CH3, CH4, for the heavy chain.

The term “chimeric antibody” means an antibody wherein the sequences for the variable regions of the light chains and of the heavy chains belong to a species that is different from that for the sequences of the constant regions of the light chains and of the heavy chains. For the purposes of the invention, the sequences for the variable regions of the heavy and light chains are preferably of murine origin, while the sequences for the constant regions of the heavy and light chains belong to a non-murine species. In this regard, for the constant regions, any non-murine mammalian species could be used, and in particular of the human, ape, porcine (Suidae), bovine, equine, feline, canine or in fact bird species; this list is not exhaustive. Preferably, the chimeric antibodies in accordance with the invention contain sequences for the constant regions of the heavy and light chains of human origin and sequences for the variable regions of the heavy and light chains of murine origin.

The term “humanized antibody” means an antibody for which all or a portion of the sequences for the regions involved in recognition of the antigen (the hypervariable regions or CDR: Complementarity Determining Region) and sometimes certain amino acids of the FR regions (Framework Regions) are of non-human origin, while the sequences for the constant regions and for the variable regions not involved in recognition of the antigen are of human origin.

The term “human antibody” means an antibody containing only human sequences, both for the variable and for the constant regions of the light chains and for the variable and constant regions of the heavy chains.

The term “antibody fragment” means any part of an immunoglobulin obtained by enzymatic digestion or obtained by bioproduction comprising at least one disulfide bridge, for example Fab, Fab′, F(ab′)₂, Fab′-SH, scFv-Fc or Fc.

The enzymatic digestion of immunoglobulins by papain generates two identical fragments which are known as Fab fragments (Fragment antigen binding), and a Fc fragment (Fragment crystallizable). The enzymatic digestion of immunoglobulins by pepsin generates a fragment F(ab′)2 and a fragment Fc split into several peptides. F(ab′)2 is formed by two Fab′ fragments linked by intercatenary disulfide bridges. Fab portions are constituted by variable regions and the CH1 and CL domains. The Fab′ fragment is constituted by the Fab region and by a hinge region. Fab′-SH refers to a Fab′ fragment in which the cysteine residue of the hinge region carries a free thiol group. The scFv (single chain Fragment variable) is a fragment obtained by engineering proteins and which is constituted solely by the variable domains VH and VL. The structure is stabilized by a short flexible peptide arm, known as a linker, which is located between the two domains. The scFv fragment may be linked to a Fc fragment in order to provide a scFv-Fc.

The term “CD56” designates “Cluster Differentiation 56”, which is a membrane glycoprotein that is a member of the immunoglobulin (Ig) superfamily containing 5 domains of the Ig type and two fibronectin type 3 domains in its extracellular portion. CD56 is also frequently known as “Neural-Cell Adhesion Molecule 1 (NCAM 1)”.

The term “CD56+ cancer” or “CD56 positive cancer” designates a cancer expressing CD56. In particular, the term “CD56+ cancer” designates any case of cancer in which the cancer cells present an expression of CD56. An expression of CD56 is frequently observed in neuroendocrinal carcinomas, pediatric tumors and certain hemopathies. It has also been reported in certain melanomas and soft tissue sarcomas. Preferably, in the context of the present invention, the CD56+ cancer is selected from melanoma, blastemal tumors, hemopathies, such as acute myeloid leukemias, myelomas, blastic plasmacytoid dendritic cell neoplasms and neuroendocrinal carcinomas such as small cell lung carcinoma and Merkel cell carcinoma. In a preferred embodiment, the CD56+ cancer is a carcinoma selected from neuroendocrinal carcinomas such as small cell lung carcinoma or Merkel cell carcinoma, preferably Merkel cell carcinoma.

In the context of the present invention, “identity” or “homology” is calculated by comparing two sequences aligned in a comparison window. The alignment of the sequences means that the number of positions (nucleotides or amino acids) that are common to the two sequences in the comparison window can be determined. The number of positions in common is therefore divided by the total number of positions in the comparison window and multiplied by 100 in order to obtain the percentage identity. The determination of the percentage identity of the sequence may be carried out manually or with the aid of well-known computer programs.

In a particular embodiment of the invention, the identity or the homology corresponds to at least one substitution, for example 1, 2, 3, 4, 5, 6, 7, 8, 9, 10 substitutions, of an amino acid residue, preferably at least one substitution of an amino acid residue carried out in a conservative manner. The “substitution of an amino acid residue carried out in a conservative manner” consists of replacing an amino acid residue by another amino acid residue having a side chain possessing similar properties. The famines of amino acids possessing side chains with similar properties are well known; examples that may be cited are basic side chains (for example lysine, arginine, histidine), acidic side chains (for example aspartic acid, glutamic acid), polar, uncharged side chains (for example glycine, asparagine, glutamine, serine, threonine, tyrosine, cysteine), apolar side chains (for example glycine, cysteine, alanine, valine, leucine, isoleucine, proline, phenylalanine, methionine, tryptophan), beta-branched side chains (for example threonine, valine, isoleucine) and aromatic side chains (for example tyrosine, phenylalanine, tryptophan, histidine).

The antibody or antibody fragment homologs or “antibody or antibody fragment variants” (i.e. antibodies or antibody fragments having the same function) therefore possess certain amino acids that may be substituted with other amino acids in the constant regions and/or variable regions, without losing the capacity for binding to the antigen. It is preferable for this substitution to be carried out within the DNA sequence that codes for the antibody or antibody fragment, i.e. the substitution is conservative in nature. The person skilled in the art will use his/her general knowledge in order to determine the number of substitutions that may be carried out and their location in order to be able to conserve the function of the antibody or of the antibody fragment. In order to determine the capacity of one or more antibody or antibody fragment variants to bind specifically to an antigen, a number of appropriate methods which are well known to the person skilled in the art and which have been described in the prior art may be used. The antibodies or the antibody fragments may therefore be tested using binding methods such as, for example, the ELISA method, the affinity chromatography method, etc. The antibody or antibody fragment variants may be generated, for example, using the “phage display” method, enabling a phage library to be generated. A large number of methods are known in order to generate a “phage display” library and target the antibody or antibody fragment variants with the desired functional characteristics.

The term “purified” and “isolated” when referring to an antibody in accordance with the invention means that the antibody is present in the substantial absence of other biological macromolecules of the same type. The term “purified” as used here preferably signifies at least 75% by weight, more preferably at least 85% by weight, yet more preferably at least 95% by weight, and most preferably at least 98% by weight of antibody with respect to the totality of the macromolecules present.

The term “pharmaceutically acceptable” means approved by a federal or state regulatory agency or listed in the American or European pharmacopeia, or in another generally recognized pharmacopeia intended to be used in animals or human beings. A “pharmaceutical composition” designates a composition comprising a pharmaceutically acceptable vehicle. As an example, a pharmaceutically acceptable vehicle may be a diluent, an adjuvant, an excipient or a vehicle with which the therapeutic agent is administered. These vehicles may be sterile liquids, such as water and oils, including those of oil, animal, vegetable or synthetic origin, such as peanut oil, soy oil, mineral oil, sesame oil, etc. Water is a preferred vehicle when the pharmaceutical composition is administered intravenously. Saline solutions and aqueous dextrose and glycerol solutions may also be used as liquid vehicles, in particular for injectable solutions. Pharmaceutically acceptable excipients include starch, glucose, lactose, saccharose, sodium stearate, glycerol monostearate, talc, sodium chloride, skimmed milk powder, glycerol, propylene glycol, water, ethanol and the like. When the pharmaceutical composition is suitable for oral administration, the tablets or capsules may be prepared using conventional means with pharmaceutically acceptable excipients such as binders (for example pre-gelatinized corn starch, polyvinylpyrrolidone or hydroxypropyl methylcellulose); fillers (for example lactose, microcrystalline cellulose or calcium hydrogen phosphate); lubricants (for example, magnesium stearate, talc or silica); disintegrants (for example potato starch or sodium starch glycolate); or wetting agents (for example, sodium lauryl sulfate). The tablets may be coated using methods that are well known in the prior art. The liquid preparations for oral administration may take the form of solutions, syrups or suspensions, for example, or may be presented in the form of a dry product for reconstitution with water or another appropriate vehicle before use. Liquid preparations of this type may be prepared using conventional means with pharmaceutically acceptable vehicles such as suspension agents (for example a sorbitol syrup, cellulose derivatives or edible hydrogenated fats); emulsifying agents (for example, lecithin or acacia); non-aqueous vehicles (for example almond oil, oily esters, ethyl alcohol or fractionated vegetable oils); and preservatives (for example sorbic acid or methyl or propyl p-hydroxybenzoates). The pharmaceutical compositions may also contain buffer salts, flavorings, colorants and sweeteners, depending on the case. The composition in accordance with the invention is preferably a pharmaceutical composition.

The term “treat” or “treatment” encompasses any beneficial effect or desirable effect on a pathology or a pathological state and may even include a minimal reduction in one or more measurable markers of the pathology or of the pathological condition. The treatment may, for example, involve either the reduction or improvement in symptoms of the pathology or the pathological state, or slowing of the progression of the disease or the pathological state. The term “treatment” does not necessarily mean the complete eradication or healing of the disease, nor of the associated symptoms.

The term “native mass spectrometry” means a mass spectrometric analysis carried out under conditions that do not denature the proteins, meaning that non-covalent bonds can be conserved. Native mass spectrometry methods have been widely described in the literature, for example in reference [4].

Antibody-Drug Conjugate

The invention concerns an antibody-drug conjugate with the following formula (I):

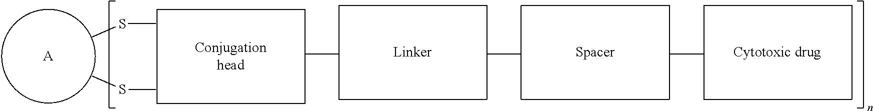

in which:
A is an anti-CD56 antibody or an antibody fragment;
the attachment head is represented by one of the following formulae:

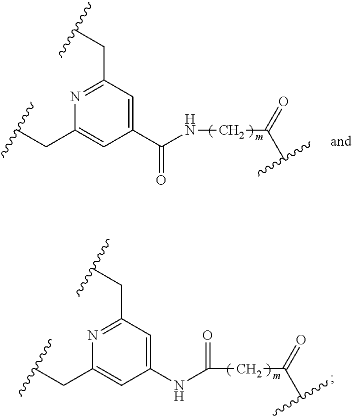

the linker is a cleavable linker selected from the following formulae:

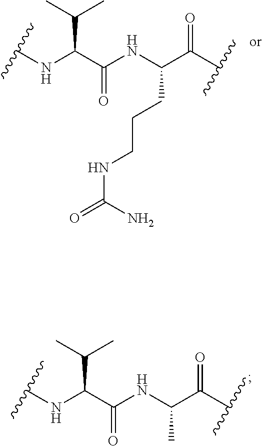 [SMILES: *N[C@H](C(=O)N[C@@H](CCCNC(N)=O)C(*)=O)C(C)C ; *N[C@H](C(=O)N[C@@H](C)C(*)=O)C(C)C]

the spacer is represented by the following formula:

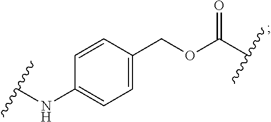 [SMILES: *Nc1ccc(COC(*)=O)cc1]

m is an integer from 1 to 10, advantageously from 2 to 7, from 3 to 6, advantageously equal to 4 or 5;
n is an integer from 1 to 4.

The anti-CD56 antibody or antibody fragment in accordance with the invention may be of mammalian origin (for example human or mouse), humanized or chimeric. It is preferably a monoclonal antibody produced in a recombinant manner by genetically modified cells using techniques that have been widely described in the prior art.

When A is an anti-CD56 antibody, it is preferably an IgG, for example IgG1, IgG2, IgG3 or IgG4.

In a particular embodiment, A is an anti-CD56 antibody or an antibody fragment that comprises:
a variable domain of a light chain comprising a CDR1 of the amino acid sequence SEQ ID NO: 1, a CDR2 of the amino acid sequence SEQ ID NO: 2, and a CDR3 of the amino acid sequence SEQ ID NO: 3; and
a variable domain of a heavy chain comprising a CDR1 of the amino acid sequence SEQ ID NO: 4, a CDR2 of the amino acid sequence SEQ ID NO: 5, and a CDR3 of the amino acid sequence SEQ ID NO: 6.

In this particular embodiment, the variable domain of the light chain has at least 80% homology, preferably at least 90% homology, for example at least 95% homology, at least 96%, at least 97%, at least 98%, at least 99% or even 100% homology with the amino acid sequence SEQ ID NO: 15; and the variable domain of the heavy chain has at least 80% homology, preferably at least 90% homology, for example at least 95% homology, at least 96%, at least 97%, at least 98%, at least 99% or even 100% homology with the amino acid sequence SEQ ID NO: 16.

Thus, A may be an antibody or an antibody fragment with the amino acid sequence SEQ ID NO: 15 for the variable domain of the light chain and with the amino acid sequence SEQ ID NO: 16 for the variable domain of the heavy chain.

In a particular embodiment, A is an antibody with the amino acid sequence SEQ ID NO: 7 for the light chain and with the amino acid sequence SEQ ID NO: 8 for the heavy chain. The heavy chain with the amino acid sequence SEQ ID NO: 8 may also have an additional lysine in the C-terminal position.

In a particularly preferred embodiment, A is the antibody with reference m906 described in the application US 2018/0214568 A1 and in reference [1]. The antibody m906 is an anti-CD56 chimeric antibody of type IgG1 with the amino acid sequence SEQ ID NO: 7 for the light chain and with the amino acid sequence SEQ ID NO: 8 for the heavy chain.

In a particular embodiment, the antibody-drug conjugate of the invention has one of the following formulae:

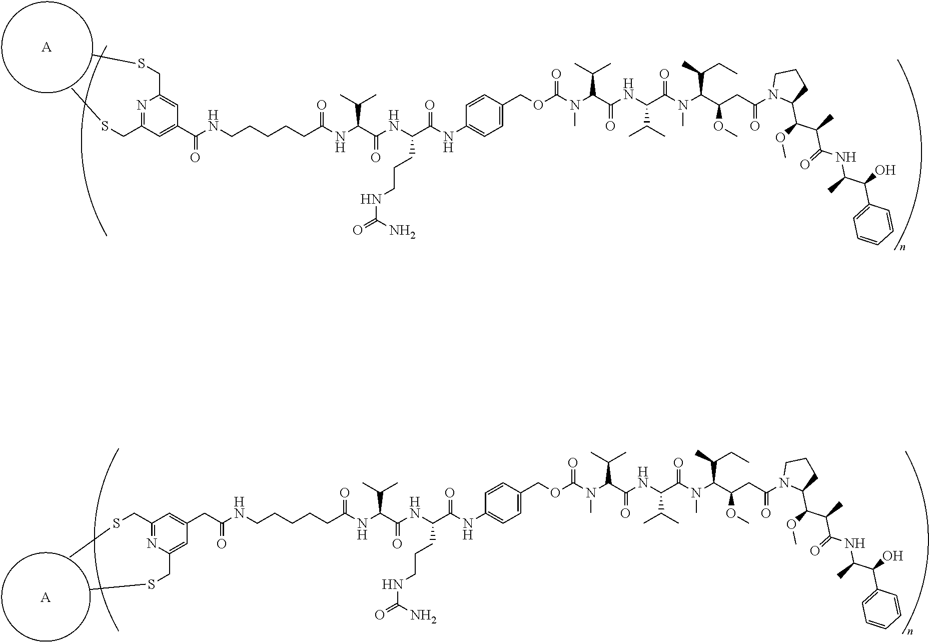

In a particular embodiment, when A is m906, the antibody-drug conjugate of the invention has the following formula (Ia):

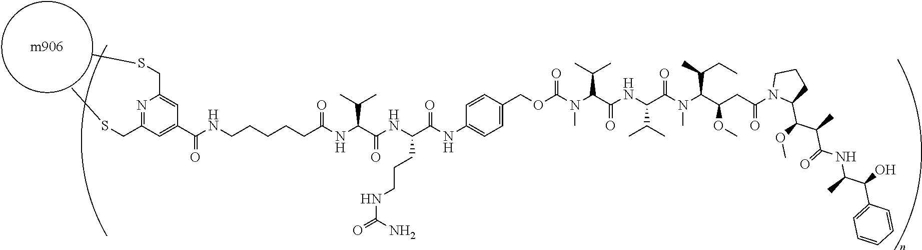

or has the following formula (Ia′):

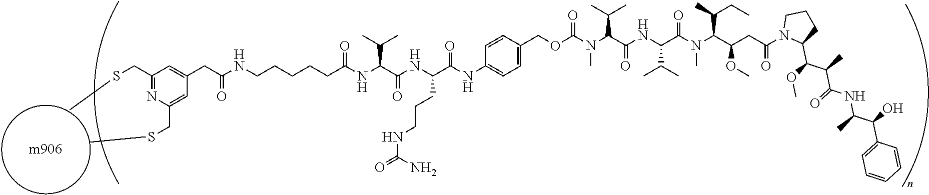

The antibody-drug conjugate identified in the examples with the reference “MF-m906-MMAE” corresponds to an antibody-drug conjugate with formula (Ia). “m906” corresponds to the antibody m906, i.e. a human anti-CD56 antibody of type IgG1 with the amino acid sequence SEQ ID NO: 7 for the light chain and with the amino acid sequence SEQ ID NO: 8 for the heavy chain.

Advantageously, the antibody-drug conjugate in accordance with the invention is purified (or isolated) by carrying out known purification techniques such as chromatographic and/or affinity column purification.

When n is equal to 1, the antibody-drug conjugate is generally termed “DAR1”. When n is equal to 2, the antibody-drug conjugate is generally termed “DAR2”. When n is equal to 3, the antibody-drug conjugate is generally termed “DAR3”. When n is equal to 4, the antibody-drug conjugate is generally termed “DAR4”.

In a particular embodiment, the antibody-drug conjugate has one or more effective functions mediated by the attenuated Fc portion. Preferably, the effective function or functions mediated by the Fc portion is/are selected from ADCC (Antibody-Dependent Cell-mediated Cytotoxicity) and CDC (Complement-Dependent Cytotoxicity). The person skilled in the art will have no difficulty in attenuating one or more effective functions mediated by the Fc portion having regard to the teaching of the prior art, for example by mutating the Fc portion. Many mutations are known to reduce the effective functions mediated by the Fc portion. It may, for example, be a mutation aimed at deglycosylating the Fc portion, in particular of suppressing glycosylation at asparagine 297.

In a particular embodiment, the antibody-drug conjugate is deglycosylated at the Fc portion, for example the antibody-drug conjugate no longer carries a glycosylation at asparagine 297.

Composition

The invention also concerns a composition comprising one or more antibody-drug conjugate(s) with formula (I), for example with formula (Ia) or with formula (Ia′), as defined above. It may be a pharmaceutical composition comprising one or more antibody-drug conjugate(s) with formula (I) as defined above, for example with formula (Ia) or with formula (Ia′), and a pharmaceutically acceptable vehicle.

The composition in accordance with the invention has the feature of being particularly homogeneous, which may result in better stability, better efficacy and/or a reduction in the secondary effects of the composition compared with a composition that is not homogeneous.

Advantageously, when A is an antibody, for example m906, the composition in accordance with the invention is characterized by the following features:

a) at least 50%, preferably at least 55%, at least 60%, at least 65%, for example at least 70%, at least 75%, at least 80%, at least 85%, at least 90%, at least 95%, at least 96%, at least 97%, at least 98% or at least 99% of the antibody-drug conjugates of the composition have an n equal to 4;
b) the mean Drug-to-Antibody Ratio (mean DAR) is comprised between 3 and 4.5, preferably comprised between 3.5 and 4, between 3.7 and 4, between 3.8 and 4, for example equal to 3.9±0.1, for example equal to 3.89. The mean DAR is generally determined by the HIC (Hydrophobic Interaction Chromatography) method or by native mass spectrometry. The HIC method and the native mass spectrometry method have been widely described in the literature. Examples that may be cited are reference [3] for the HIC method and reference [4] for the native mass spectrometry method;
c) at least 95%, preferably at least 96%, at least 97%, at least 98%, at least 99% of the antibody-drug conjugates are present in the form of the monomer. The percentage of monomer is generally determined by the SEC (Size Exclusion Chromatography) method. The SEC method has been widely described in the literature, for example in reference [2];
d) one or more of the ratios for n defined below; or
e) a combination of two, three or four features selected from a), b, c) and d).

When A is an antibody, for example m906, the composition in accordance with the invention may be characterized by the following ratios for n:
less than 5%, less than 4%, less than 3%, less than 2%, less than 1%, less than 0.75%, less than 0.5%, less than 0.25%, less than 0.2%, less than 0.1%, for example approximately 0% of the antibody-drug conjugates of the composition have an n equal to 1,
less than 10%, less than 9%, less than 8%, less than 7%, less than 6%, less than 5%, less than 4%, less than 3%, less than 2%, less than 1% of the antibody-drug conjugates of the composition have an n equal to 2,
less than 25%, less than 20%, less than 18%, less than 17% of the antibody-drug conjugates of the composition have an n equal to 3, and/or
at least 50%, at least 55%, at least 56%, at least 57%, at least 58%, at least 59%, at least 60%, at least 65%, at least 70%, at least 71%, at least 72%, at least 73%, at least 74% of the antibody-drug conjugates of the composition have an n equal to 4.

When A is an antibody, for example m906, the composition in accordance with the invention may also be characterized by the following ratios for n:
between 0% and 5%, between 0% and 2%, between 0% and 1%, between 0% and 0.75%, between 0% and 0.5%, between 0% and 0.25%, between 0% and 0.1% of the antibody-drug conjugates of the composition have an n equal to 1,
between 0% and 10%, between 0% and 7%, between 0% and 6%, between 3% and 6%, between 5% and 6%, between 0% and 5%, between 0% and 4%, between 0% and 3%, between 0% and 2%, between 0% and 1% of the antibody-drug conjugates of the composition have an n equal to 2,
between 0% and 25%, between 5% and 20%, between 10% and 20%, between 15% and 17% of the antibody-drug conjugates of the composition have an n equal to 3, and
between 50 and 85%, between 50% and 80%, between 50% and 75%, between 70% and 85%, between 70% and 80%, between 70% and 75%, between 50% and 60%, between 55% and 60%, of the antibody-drug conjugates of the composition have an n equal to 4.

The ratios for n may be determined by the HIC (Hydrophobic Interaction Chromatography) method or by native mass spectrometry.

In a first particular embodiment, when A is an antibody, for example m906, the composition in accordance with the invention may be characterized by the following ratios for n determined by the HIC (Hydrophobic Interaction Chromatography) method:
less than 5%, less than 4%, less than 3%, less than 2%, less than 1%, less than 0.75%, less than 0.5%, less than 0.25%, less than 0.2%, less than 0.1% of the antibody-drug conjugates of the composition have an n equal to 1,
less than 10%, less than 9%, less than 8%, less than 7%, less than 6%, for example approximately 5.5% of the antibody-drug conjugates of the composition have an n equal to 2,
less than 25%, less than 20%, less than 18%, less than 17%, for example approximately 16% of the antibody-drug conjugates of the composition have an n equal to 3, and/or
at least 50%, at least 55%, at least 56%, at least 57%, at least 58%, at least 59%, for example approximately 60% of the antibody-drug conjugates of the composition have an n equal to 4.

Thus, when A is an antibody, for example m906, the composition in accordance with the invention may also be characterized by the following ratios for n determined by the HIC (Hydrophobic Interaction Chromatography) method:
between 0% and 5%, between 0% and 2%, between 0% and 1%, between 0% and 0.75%, between 0% and 0.5%, between 0% and 0.25%, between 0% and 0.1% of the antibody-drug conjugates of the composition have an n equal to 1,
between 0% and 10%, between 0% and 7%, between 0% and 6%, between 3% and 6%, between 5% and 6% of the antibody-drug conjugates of the composition have an n equal to 2,
between 0% and 25%, between 5% and 20%, between 10% and 20%, between 15% and 17% of the antibody-drug conjugates of the composition have an n equal to 3, and
between 50 and 75%, between 50% and 60%, between 55% and 60%, of the antibody-drug conjugates of the composition have an n equal to 4.

In a second particular embodiment, when A is an antibody, for example m906, the composition in accordance with the invention may be characterized by the following ratios for n determined by native mass spectrometry:
less than 5%, less than 4%, less than 3%, less than 2%, less than 1%, less than 0.75%, less than 0.5%, less than 0.25%, less than 0.2%, less than 0.1%, for example approximately 0% of the antibody-drug conjugates of the composition have an n equal to 1,
less than 10%, less than 9%, less than 8%, less than 7%, less than 6%, less than 5%, less than 4%, less than 3%, less than 2%, less than 1%, for example approximately 0% of the antibody-drug conjugates of the composition have an n equal to 2,
less than 25%, less than 20%, less than 18%, less than 17% of the antibody-drug conjugates of the composition have an n equal to 3, and/or
at least 50%, at least 55%, at least 56%, at least 57%, at least 58%, at least 59%, at least 60%, at least 65%, at least 70%, at least 71%, at least 72%, at least 73%, at least 74% of the antibody-drug conjugates of the composition have an n equal to 4.

Thus, when A is an antibody, for example m906, the composition in accordance with the invention may also be characterized by the following ratios for n determined by native mass spectrometry:
between 0% and 5%, between 0% and 2%, between 0% and 1%, between 0% and 0.75%, between 0% and 0.5%, between 0% and 0.25%, between 0% and 0.1% of the antibody-drug conjugates of the composition have an n equal to 1,
between 0% and 10%, between 0% and 7%, between 0% and 6%, between 0% and 5%, between 0% and 4%, between 0% and 3%, between 0% and 2%, between 0% and 1% of the antibody-drug conjugates of the composition have an n equal to 2,
between 0% and 25%, between 5% and 20%, between 10% and 20%, between 15% and 17% of the antibody-drug conjugates of the composition have an n equal to 3, and
between 50% and 85%, between 50% and 80%, between 50% and 75%, between 70% and 85%, between 70% and 80%, between 70% and 75% of the antibody-drug conjugates of the composition have an n equal to 4. When A is an antibody, the composition in accordance with the invention may also comprise DAR0s, i.e. antibodies without a cytotoxic conjugate. Preferably, there is less than 10%, less than 9%, less than 8%, less than 7%, less than 6%, less than 5%, less than 4%, less than 3%, less than 2%, less than 1%, less than 0.5%, less than 0.4%, less than 0.2%, less than 0.1% of DAR0, for example approximately 0% of DAR0. The percentage of DAR0 may be determined by the HIC (Hydrophobic Interaction Chromatography) method or by native mass spectrometry.

The composition in accordance with the invention may also comprise DAR5, i.e. antibody-drug conjugates having 5 cytotoxic conjugates. Preferably, there is less than 25%, less than 20%, less than 15%, less than 10%, less than 9%, less than 8%, less than 7%, less than 6%, less than 5% of DAR5. The presence of DAR5 in antibody-drug conjugate compositions has been widely described in the literature, however the exact structure of the DAR5s has not been studied to any great extent. Thus, the mean DAR is calculated by taking into account all of the DARs present in the composition, i.e. DAR0, DAR1 (i.e. n=1), DAR2 (i.e. n=2), DAR3 (i.e. n=3), DAR4 (i.e. n=4), DAR5, etc. Preferably, when A is an antibody, the composition in accordance with the invention does not comprise any DARs higher than DAR5, for example DARE, DAR7, etc. The percentages of DAR5 and higher may be determined by the HIC (Hydrophobic Interaction Chromatography) method or by native mass spectrometry. In a very particular embodiment, the composition in accordance with the invention has the HIC profile of FIG. 1 or the native mass spectrometry profile of FIG. 3.

Therapeutic Use

The invention also concerns an antibody-drug conjugate with formula (I) in accordance with the invention or a composition comprising an antibody-drug conjugate with formula (I) in accordance with the invention for use as a drug, for example for use in the treatment of a CD56+ cancer such as melanoma, blastemal tumors, hemopathies, such as acute myeloid leukemias, myelomas, blastic plasmacytoid dendritic cell neoplasms and neuroendocrinal carcinomas. Advantageously, the CD56+ cancer is selected from neuroendocrinal carcinomas such as small cell lung cancer or Merkel cell carcinoma, preferably Merkel cell carcinoma.

The antibody-drug conjugate or the composition in accordance with the invention is preferably formulated for parenteral administration, for example intravascuiar (intravenous or intra-arterial), intraperitoneal or intramuscular administration. The term “administered parenterally” as used here designates modes of administration other than enteral and topical administration, generally by injection, and includes, although this is not limiting, administration that is intravascular, intravenous, intramuscular, intra-arterial, intrathapsal, intracapsular, intraorbital, intratumoral, intracardiac, intradermal, intraperitoneal, by injection, transtracheal perfusion, subcutaneous, intra-articular, subcapsular, subarachnoid, intraspinal and intrasternal. Intravenous administration is preferred in the context of the present invention, for example by intravenous perfusion.

The dose of antibody-drug conjugate administered to a subject requiring it will vary as a function of a number of factors including, although this is not limiting, the mode of administration, the type and severity of the disease being treated, the condition of the patient, the patient's obesity level, the age of the patient, etc. The person skilled in the art will readily determine the posological range required as a function of these and other factors on the basis of their knowledge in this field. The appropriate dose may also be determined with animal models or with clinical trials. As an example, typical doses of antibody-drug conjugate in accordance with the invention may be from 5 mg/m² to 250 mg/m², for example from 30 mg/m² to 150 mg/m², from 5 mg/m² to 75 mg/m², from 75 mg/m² to 120 mg/m², for example equal to 100 mg/m², 75 mg/m², 60 mg/m². The administration may be made all at once or, more generally, in several doses. The administration plan may include an initial dose then maintenance doses, for example weekly, every two weeks, every three weeks, every month, or longer. The treatment duration may vary as a function of the disease being treated and the subject.

The antibody-drug conjugate or the composition in accordance with the invention may be used in a monotherapy or in combination with drugs with a recognized therapeutic effect for the disease under consideration. It may, for example, be paclitaxel, docetaxel, doxorubicin, cyclophosphamide, lenalidomide, dexamethasone, carboplatin, etoposide, or an antibody used in anti-cancer immunotherapy, such as an anti-PD1 or anti-PD-L1.

The description also concerns a method for the treatment of a CD56+ cancer in a subject, comprising administering to the subject a therapeutically effective quantity of an antibody-drug conjugate with formula (I) in accordance with the invention or a composition comprising an antibody-drug conjugate with formula (I) in accordance with the invention.

Preparation Method

The description also concerns a method for the preparation of an antibody-drug with formula (I) as defined above, in which a cytotoxic conjugate with the following formula (II):

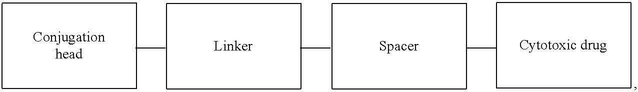

as defined above is reacted with an anti-CD56 antibody or an antibody fragment as defined above.

The invention concerns a method for the preparation of an antibody-drug conjugate in accordance with the invention, comprising the following steps:

(i) preparing a cytotoxic conjugate by coupling a attachment head with formula:

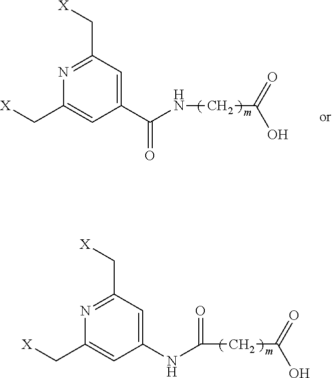

to a compound with formula:

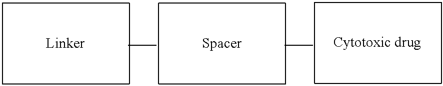

in which:
the linker is a cleavable linker selected from the following formulae:

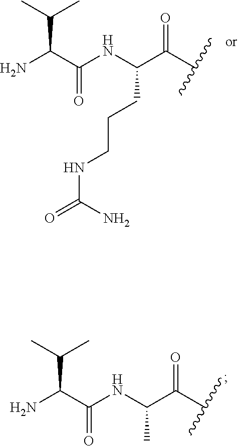 [SMILES: *C(=O)[C@H](CCCNC(N)=O)NC(=O)[C@@H](N)C(C)C ; *C(=O)[C@H](C)NC(=O)[C@@H](N)C(C)C]

the spacer is represented by the following formula:

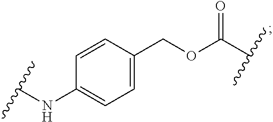 [SMILES: *Nc1ccc(COC(*)=O)cc1]

### X is Br, Cl, I or F;

m is an integer from 1 to 10, advantageously from 2 to 7, from 3 to 6, advantageously equal to 4 or 5; and
(ii) reacting the cytotoxic conjugate obtained in step (i) with an anti-CD56 antibody or an anti-CD56 antibody fragment. The attachment head is described in more detail in the definition of “cytotoxic conjugate” and in the “antibody-drug conjugate” section above, with the difference that the attachment head employed in the method comprises a terminal carboxylic acid function.

The linker is described in more detail in the definition of “cytotoxic conjugate” and in the “antibody-drug conjugate” section above, with the difference that the linker employed in the method comprises a terminal amine function.

The spacer is described in more detail in the definition of “cytotoxic conjugate” and in the “antibody-drug conjugate” section above.

The cytotoxic drug is described in more detail in the definition of “cytotoxic conjugate” and in the “antibody-drug conjugate” section above.

The anti-CD56 antibody is described in more detail in the definition of “cytotoxic conjugate” and the “antibody-drug conjugate” section above.

When the cytotoxic drug is MMAE, step (i) can be used to obtain a cytotoxic conjugate with formula (IIa) or (IIa′).

In a particular embodiment, the method for the preparation of a cytotoxic conjugate in accordance with step (i) comprises a step that consists of coupling 6-(2,6-bis(bromomethyl)pyridin-4-yl)amidohexanoic acid or 6-((2,6-bis(bromomethyl)pyridin-4-yl)amino)-6-oxohexanoic acid with MMAE valine-citrulline-p-aminobenzoyl carbamate or a salt of this compound. In this particular embodiment, the method for the preparation of a cytotoxic conjugate in accordance with the invention can be used to obtain MMAE 6-(2,6-bis(bromomethyl)pyridin-4-yl)amido-N-hexanamide-valine-citrulline-p-aminobenzoyl carbamate or MMAE 6-((2,6-bis(bromomethyl)pyridin-4-yl)amino)-6-oxohexanamide-valine-citrulline-p-aminobenzoyl carbamate, respectively.

In a particular embodiment, the method for the preparation of an antibody-drug conjugate in accordance with the invention comprises a step that consists of reacting MMAE 6-(2,6-bis(bromomethyl)pyridin-4-yl)amido-N-hexanamide-valine-citrulline-p-aminobenzoyl carbamate or MMAE 6-((2,6-bis(bromomethyl)pyridin-4-yl)amino)-6-oxohexanamide-valine-citrulline-p-aminobenzoyl carbamate with an anti-CD56 antibody or an anti-CD56 antibody fragment.

### BRIEF DESCRIPTION OF THE FIGURES

FIG. 1 represents the HIC (Hydrophobic Interaction Chromatography) profile of an antibody-drug conjugate composition in accordance with the invention. The figure shows that the composition is enriched in DAR4, with more than 50% DAR4.

FIG. 2 represents a SEC (Size Exclusion Chromatography) analysis of an antibody-drug conjugate composition in accordance with the invention. The figure shows that the composition is extremely homogeneous, with more than 99% of monomer.

FIG. 3 represents a native mass spectrometric analysis on a Vion IMS Qtof mass spectrophotometer coupled to an Acquity UPLC H-Class system from Waters (Wilmslow, UK) of an antibody-drug conjugate composition in accordance with the invention. This method can be used to clearly identify each type of DAR. The figure shows that the composition is enriched in DAR4, with close to 75% DAR4.

FIG. 4 represents the expression of CD56 for different cell lines (4 of MCC and one breast cancer control line) by flow cytometry after incubation with the anti-CD56 antibody labeled with the fluorophore FITC (Fluorescein IsoThioCyanate).

FIG. 5A, FIG. 5B, FIG. 5C, FIG. 5D, FIG. 5E represent the evaluation of the performances of the ADC MF-m906-MMAE, compared with the m906 controls (non-coupled antibody), MF-TTZ-MMAE (ADC Trastuzumab coupled to MMAE) and MMAE (toxin alone) on the MCC cell lines (FIG. 5A: WaGa, FIG. 56: PeTa, FIG. 5C: MS-1 and FIG. 5D: MKL-2) and breast cancer cell line (FIG. 5E: SK-BR3). The experiments were carried out independently in duplicate (6 biological replicates/experiment). The results are expressed as the mean (+/−SEM) of the percentage obtained.

FIG. 6A-6C represent the evaluation of the specificity of MF-m906-MMAE in the WaGa lines after knock-down of CD56. The WaGa cells were transduced independently with lentiviral vectors containing two distinct shRNAs inducible by doxycycline (Dox) (A, B and C) and targeting the sequences of CD56. After antibiotic selection (puromycin), the cells were exposed to doxycycline for 7 days before evaluation. (A-B-C): Confirmation of knock-down of CD56 by real-time RT-qPCR (A) (*: p<0.05, the control cells without doxycycline were used as a reference), by Western blot (attenuated mass of CD56=140 KDa) (B). (C) Evaluation of the cytotoxic effect of MF-m906-MMAE on cells expressing CD56 (−Dox) or not expressing CD56 (+Dox) by using three different shRNAs (Sh RNA A, Sh RNA B and Sh RNA C). The experiments were carried out independently in duplicate (6 biological replicates/experiment). The results are expressed as the mean (+/−SEM) of the percentage obtained.

FIG. 7 illustrates the evaluation of the performances of the conjugate MF-m906-MMAE and the controls (PBS and MF-TTZ-MMAE, a Trastuzumab ADC coupled to MMAE) in a xenograft model of MCC. FIG. 7 represents the plots for relative tumor volume (mean+/−SEM) during the study. The relative tumor volumes (RTV) were calculated using the tumor volume for the start of the study as the reference (Volume at d0=100%). Each point represents the mean of the RTVs for the groups treated with MF-m906-MMAE, MF-TTZ-MMAE and PBS, the gray arrows indicating the time of the injections.

FIG. 8 illustrates the evaluation of the performances of the conjugate MF-m906-MMAE and the controls (PBS and MF-TTZ-MMAE, a Trastuzumab ADC coupled to MMAE) in a xenograft model of MCC. FIG. 8 represents the weight of the tumors at the end of the study in the experimental group and the control groups (*: p<0.05). The horizontal lines are the means, the quartiles and the limits.

FIG. 9 represents the evaluation of the performances of the ADC MF-m906-MMAE, compared with the controls m906 (non-coupled antibody), MF-TTZ-MMAE (Trastuzumab ADC coupled to MMAE) and MMAE (toxin alone) on the cell line for small cell lung cancer, H69. The experiments were carried out independently in triplicate (6 biological replicates/experiment). The results are expressed as the mean (+/−SEM) of the percentage obtained.

### EXAMPLES

### Example 1: Synthesis of a Cytotoxic Conjugate in Accordance with the Invention

### General Reaction Scheme

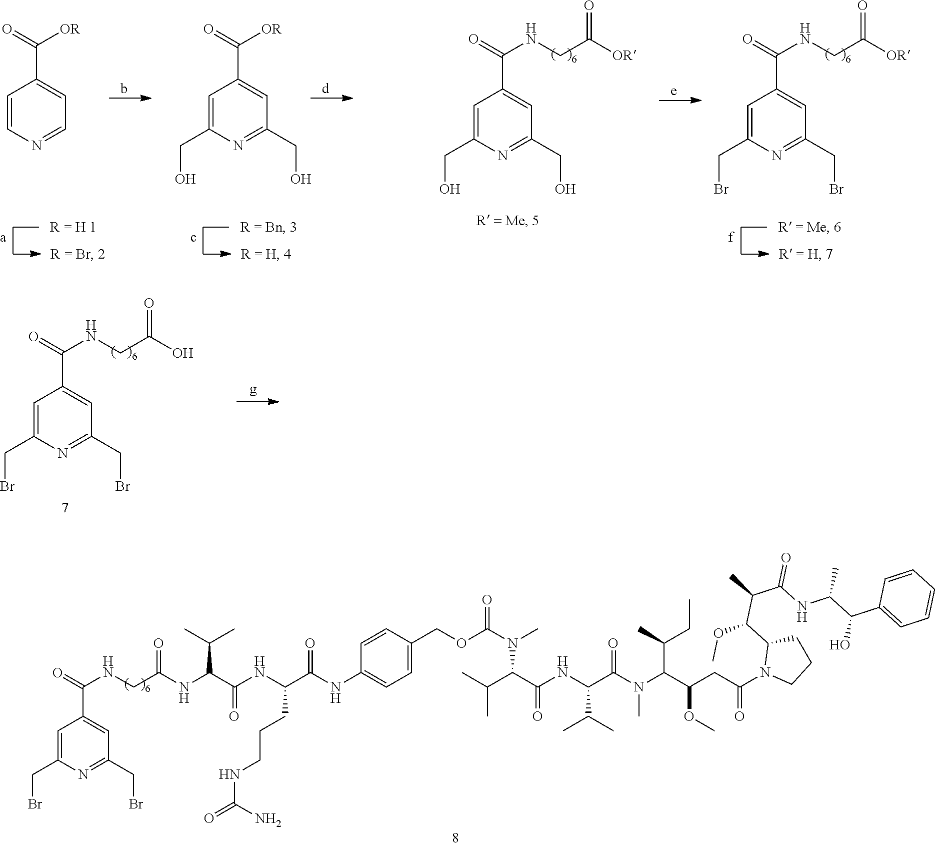

(a) BnOH, SOCl₂, DCM, 18 h, 75° C.; (b) APS, MeOH, H₂O, H₂SO₄, 1 h, 50° C.; (c) H₂, Pd/C, MeOH, 2 h, TA; (d) HATU, 2,6-lutidine, DMF, H₂N—(CH₂)₅—COOMe, 15 h, 0° C., then TA; (e) PBr₃, MeCN, 2h, 45° C.; (f) LiOH, THF, H₂O, 8.5 h, TA; (g) EEDQ, DIPEA, DMF, MeCN, H₂N-VCPABC-MMAE, 2 h20, 25° C.

Detailed Reaction Scheme

### Preparation of benzyl isonicotinate (2)

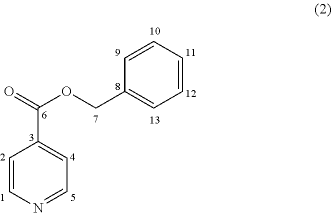 [SMILES: O=C(OCc1ccccc1)c1ccncc1]

Isonicotinic acid (1) (5.00 g; 40.614 mmol; 1.0 eq) was dissolved in thionyl chloride (15 mL; 206.77 mmol; 5.1 eq) and heated under reflux overnight. After returning to ambient temperature, the excess thionyl chloride was eliminated by evaporation under reduced pressure, then the residue obtained was dissolved in anhydrous dichloromethane (55 mL). Benzyl alcohol was added (4.2 mL; 40.614 mmol; 1.0 eq) and the mixture was stirred under reflux for 10 h. After returning to ambient temperature, the reaction medium was neutralized with a saturated solution of sodium hydrogen carbonate and extracted with dichloromethane (3×100 mL). The organic phases were combined, washed with a saturated solution of sodium chloride, dried over magnesium sulfate and concentrated under reduced pressure. The product obtained was purified by flash chromatography (SiO₂, cyclohexane/ethyl acetate 50:50) in order to give (2) (6.97 g; 80%) in the form of a colorless oil.

¹H NMR (300 MHz, DMSO) δ 8.80 (dd; J=6.1; 1.6 Hz; 2H1,5), 7.86 (dd; J=6.1; 1.6 Hz, 2H2,4), 7.56-7.29 (m; 5H9-13), 5.39 (s; 2H7).

¹³C NMR (75 MHz, DMSO) δ 165.0 (1C₆); 151.3 (2C_{1,5}); 137.2 (1C₃); 136.1 (1C₈); 129.0 (2C_{10,12}); 128.8 (1C₁₁); 128.6 (2C_{9,13}); 123.0 (2C_{2,4}); 67.4 (1C₇).

HRMS (ESI): calculated neutral mass for C₁₃H₁₁NO₂ [M]: 213.0790; observed: 213.0796.

### Preparation of benzyl 2,6-bis(hydroxymethyl)isonicotinate (3)

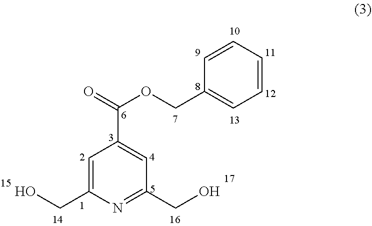 [SMILES: O=C(OCc1ccccc1)c1cc(CO)nc(CO)c1]

Benzyl isonicotinate (2) (2.48 g; 11.630 mmol; 1.0 eq) was dissolved in methanol (43 mL), stirred at 50° C. and concentrated sulfuric acid (320 μL; 6.016 mmol; 0.52 eq) was added. A solution of ammonium persulfate (26.500 g; 116.000 mmol; 10.0 eq) in water (43 mL) was added in two steps: a first rapid addition of 30 droplets; a white suspension was formed, then rapidly, drop by drop, for 5 min. The reaction ran away up to 75° C., then the yellow solution obtained was stirred at 50° C. for an additional 1 h. After returning to ambient temperature, the methanol was evaporated under reduced pressure. 50 mL of ethyl acetate was added and the medium was neutralized by addition of a saturated solution of sodium hydrogen carbonate. The aqueous phase was extracted with ethyl acetate (3×100 mL) and the combined organic phases were washed with a saturated solution of sodium chloride, dried over magnesium sulfate, then concentrated under reduced pressure. The impure product was purified by flash chromatography (SiO₂, dichloromethane/methanol, 95:5) in order to give (3) (1.56 g; 49%) in the form of a beige solid.

¹H NMR (300 MHz, DMSO) δ 7.81 (s; 2H_{2,4}); 7.55-7.32 (m; 5H₉₋₁₃); 5.60 (t; J=5.9 Hz; 2H_{15,17}); 5.40 (s; 2H₇); 4.59 (d; J=5.9 Hz; 4H14,16).

¹³C NMR (75 MHz, DMSO) δ 165.0 (1C6); 162.8 (2C_{1,5}); 138.0 (1C₃); 135.7 (1C₈); 128.6 (2C_{10,12}); 128.4 (1C₁₁); 128.3 (2C_{9,13}); 117.0 (2C_{2,4}); 66.9 (1C₇); 63.9 (2C_{14,16}).

HRMS (ESI): calculated neutral mass for C₁₅H₁₅NO₄ [M]: 273.1001; observed: 273.1001.

### Preparation of 2,6-bis(hydroxymethyl)isonicotinic acid (4)

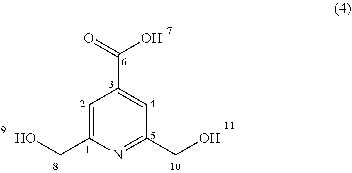 [SMILES: O=C(O)c1cc(CO)nc(CO)c1]

Benzyl 2,6-bis(hydroxymethyl)isonicotinate (3) (1.33 g; 4.867 mmol; 1.0 eq) was dissolved in methanol (50 mL) and the solution was degassed with argon for 15 min. Palladium on carbon, 10% by weight (133 mg) was added and the reaction medium was stirred at ambient temperature in an atmosphere of hydrogen for 2 h. The reaction medium was filtered over dicalite (rinsed with methanol). The filtrate was concentrated under reduced pressure in order to give (4) (849 mg; 95%) in the form of a beige solid.

¹H NMR (300 MHz, DMSO) δ 7.78 (s; 2H_{2,4}); 5.54 (s broad; 2H_{9,11}); 4.59 (s; 4H_{8,10}).

¹³C NMR (75 MHz, DMSO) δ 166.7 (106); 162.5 (2C_{1,5}); 139.4 (1C₃); 117.3 (2C_{2,4}); 64.0 (2C_{8,10}).

HRMS (ESI): calculated neutral mass for C81-19N04 [M]y 183.0532; observed: 183.0526.

### Preparation of methyl 6-((2,6-bis(hydroxymethyl)pyridin-4-yl)amidohexanoate (5)

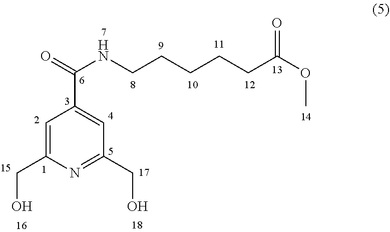 [SMILES: COC(=O)CCCCCNC(=O)c1cc(CO)nc(CO)c1]

2,6-bis(hydroxymethyl)isonicotinic acid (4) (50 mg; 0.273 mmol; 1 eq) was dissolved in anhydrous N,N-dimethylformamide (3.0 mL), the solution was cooled to 0° C., then HATU (156 mg; 0.410 mmol; 1.5 eq) and 2,6-lutidine (147.0 μL; 1.260 mmol; 4.7 eq) were added. The activation solution was stirred at 0° C. for 15 min, then methyl 6-aminohexanoate (59 mg; 0.322 mmol; 1.2 eq) was added. The walls of the flask were rinsed with 2 mL of anhydrous N,N-dimethylformamide and the reaction medium was stirred at ambient temperature for 15 h. The reaction mixture was diluted in ethyl acetate, washed three times with a saturated solution of sodium chloride, dried over magnesium sulfate, filtered and concentrated under reduced pressure. The product was purified by flash chromatography (dichloromethane/methanol, 90:10) in order to give (5) (76 mg; 91%) in the form of an off-white solid.

¹H NMR (300 MHz, DMSO) δ 8.79 (t; J=5.6 Hz; 1H₇); 7.71 (s; 2H_{2,4}); 5.50 (t; J=5.8 Hz; 2H_{16,18}); 4.57 (d; J=5.8 Hz; 4H_{15,17}); 3.57 (s; 3H₁₄); 3.25 (m; 2H₈); 2.30 (t; J=7.4 Hz; 2H₁₂); 1.62-1.45 (m; 4H_{9,11}); 1.37-1.21 (m; 2H₁₀).

¹³C NMR (75 MHz, DMSO) δ 173.3 (1C₁₃); 165.1 (1C₆); 161.8 (2_{1,5}); 142.9 (1C₃); 115.8 (2C_{2,4}); 64.1 (2C_{15,17}); 51.2 (1C₁₄); 38.5 under DMSO peak (1C₈); 33.2 (1C₁₂); 28.6 (1C₉); 25.9 (1C₁₁); 24.2 (1C₁₀).

HRMS (ESI): calculated neutral mass for C15H₂₂N₂O₅ [M]: 310.1529; observed: 310.1526.

### Preparation of methyl 6-(2,6-bis(bromomethyl)pyridin-4-yl)amidohexanoate (6)

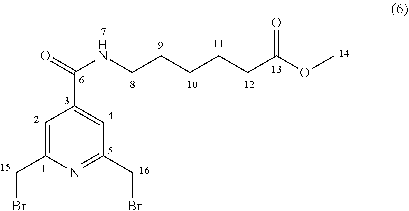 [SMILES: COC(=O)CCCCCNC(=O)c1cc(CBr)nc(CBr)c1]

Methyl 6-((2,6-bis(hydroxymethyl)pyridin-4-yl)amidohexanoate (5) (55 mg; 0.177 mmol; 1 eq) was taken up into suspension in anhydrous acetonitrile (10.5 mL) then phosphorus tribromide (50 μL; 0.532 mmol; 3.0 eq) was added dropwise. The reaction medium was stirred at 45° C. for 2 h. The solution was cooled to 0° C., neutralized with water (10 mL) and extracted with ethyl acetate (3×15 mL). The combined organic phases were washed with a saturated solution of sodium chloride, dried over magnesium sulfate and concentrated under reduced pressure. The product was purified by flash chromatography (SiO₂, cyclohexane/ethyl acetate 60:40) in order to give (6) (57 mg; 74%) in the form of a white solid.

¹H NMR (300 MHz; DMSO) δ 8.83 (t, J=5.6 Hz; 1H₇); 7.84 (s; 2H_{2,4}); 4.74 (s; 4H_{15,16}); 3.57 (s, 3H₁₄); 3.31-3.20 (m; 2H₈); 2.31 (t; J=7.4 Hz; 2H₁₂); 1.64-1.45 (m; 4H_{9,11}); 1.39-1.22 (m, 2H₁₀).

¹³C NMR (75 MHz, DMSO) δ 173.3 (1C₁₃); 163.8 (1C₆); 157.5 (2C_{1,5}); 144.2 (1C₃); 120.8 (2C_{2,4}); 51.2 (1C₁₄); 38.9 under DMSO peak (1C₈); 34.1 (2C_{15,16}); 33.2 (1C₁₂); 28.5 (1C₉); 25.9 (1C₁₁); 24.2 (1C₁₀).

HRMS (ESI): calculated neutral mass for C₁₅H₂₀Br₂N₂O₃ [M]: 433.9841; observed: 433.9832.

### Preparation of 6-(2,6-bis(bromomethyl)pyridin-4-yl)amidohexanoic acid (7)

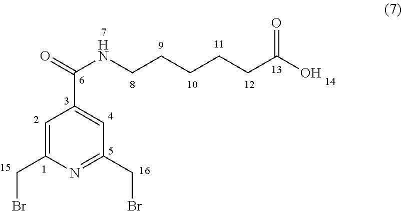 [SMILES: O=C(O)CCCCCNC(=O)c1cc(CBr)nc(CBr)c1]

Methyl 6-(2,6-bis(bromomethyl)pyridin-4-yl)amidohexanoate (6) (57 mg; 0.131 mmol; 1.0 eq) was dissolved in tetrahydrofuran (4 mL) and a solution of hydrated lithium hydroxide (8 mg; 0.327 mmol; 2.5 eq) in water (4 mL) was added slowly. The reaction medium was stirred at ambient temperature for 8.5 h. The tetrahydrofuran was evaporated off under reduced pressure and the aqueous residue was treated with an aqueous 1N hydrochloric acid solution and extracted with ethyl acetate (3×10 mL). The combined organic phases were washed with a saturated solution of sodium chloride, dried over magnesium sulfate and concentrated under reduced pressure. The product was purified by flash chromatography (dichloromethane/methanol, 90:10) in order to give (7) (44 mg; 80%) in the form of a white solid.

¹H NMR (300 MHz; DMSO) δ 12.00 (s; 1H₁₄); 8.83 (t; J=5.5 Hz; 1H₇); 7.84 (s; 2H_{2,4}); 4.74 (s; 4H_{15,17}); 3.31-3.21 (m; 2H₈); 2.21 (t; J=7.3 Hz; 2H₁₂); 1.60-1.46 (m; 4H_{9,11}); 1.39-1.25 (m; 2H₁₀).

¹³C NMR (75 MHz; DMSO) δ 174.4 (1C₁₃); 163.8 (1C₆); 157.5 (2C_{1,5}); 144.1 (1C₃); 120.8 (2C_{2,4}); 39.0 under DMSO peak (1C₈); 34.1 (2C_{15,16}); 33.6 (1C₁₂); 28.6 (1C₉); 26.0 (1C₁₁); 24.2 (1C₁₀).

HRMS (ESI): m/z calculated for C₁₄H₁₉Br₂N₂O₃ [M+H]⁺: 420.9757; observed: 420.9752.

### Preparation of MMAE 6-(2,6-bis(bromomethyl)pyridin-4-yl)amido-N-hexanamide-valine-citrulline-p-aminobenzoyl carbamate (8)

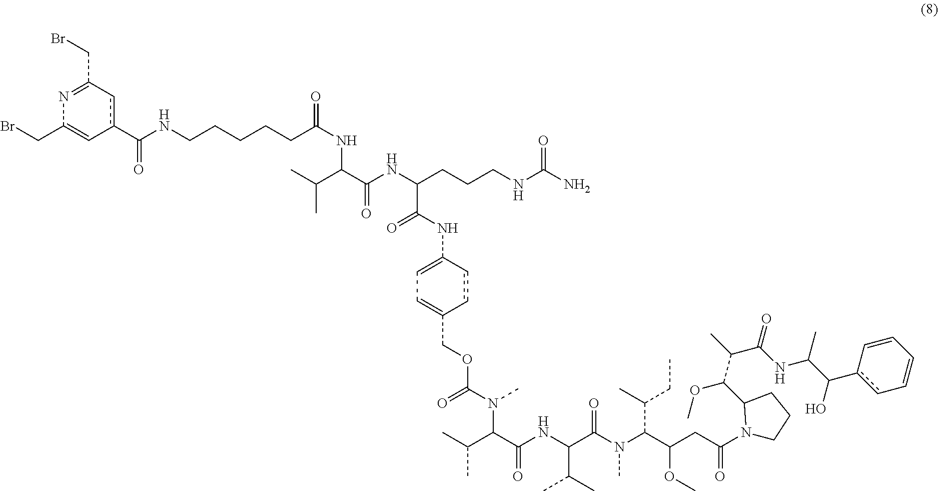

In an inert atmosphere, in darkness and under anhydrous conditions, 6-(2,6-bis(bromomethyl)pyridin-4-yl)amidohexanoic acid (7) (13.2 mg; 0.0313 mmol; 2.28 eq) was dissolved in anhydrous acetonitrile (1.2 mL), then N-ethoxycarbonyl-2-ethoxy-1,2-dihydroquinoline (EEDQ) (21.2 mg; 0.0857 mmol; 6.25 eq) was added. The activation medium was stirred under 25° C. for 1 h 20. A solution of the trifluoroacetic acid salt of MMAE valine-citrulline-p-aminobenzoyl carbamate (17.0 mg; 0.0137 mmol; 1.0 eq), dissolved in anhydrous N,N-dimethylformamide (300 μL) in the presence of N,N-diisopropylethylamine (9.4 μL; 0.0537 mmol; 3.92 eq), was added to the activation medium. The reaction medium obtained was stirred at 25° C. for 1 h. The mixture was diluted 2-fold with N,N-dimethylformamide and purified by semi-preparative high pressure liquid chromatography (t_{R}=22.1 min; on the Gilson PLC 2050 system [ARMEN V2 (pump) and ECOM TOYDAD600 (UV detector)], UV detection at 254 nm at 25° C.; Waters XBridge™ C-18 column; 5 μm (250 mm×19.00 mm); elution carried out with 0.1% of trifluoroacetic acid (by volume) in water (solvent A), and acetonitrile (solvent B); gradient 20% to 100% of B for 32 min, then 100% of B for 6 min at 17.1 mL/min) in order to give (8) (18.2 mg; 87%) in the form of a white solid.

¹H NMR (300 MHz, DMSO) δ (ppm) 10.04-9.95 (m; 1H); 8.94-8.79 (m; 1H); 8.20-8.06 (m; 2H); 7.98-7.87 (m; 1H); 7.84 (s; 2H); 7.81 (s; 1H); 7.70-7.61 (m; 1H); 7.58 (d; J=8.2 Hz; 2H); 7.38-7.11 (m; 6H); 6.07-5.92 (m; 1H); 5.47-5.37 (m; 1H); 5.15-4.96 (m; 1H); 4.73 (s; 4H); 4.54-4.29 (m; 2H); 4.32-4.12 (m; 1H); 4.05-3.92 (m; 1H); 3.30-3.08 (m; 9H); 3.06-2.93 (m; 2H); 2.91-2.77 (m; 2H); 2.24-2.05 (m; 2H); 2.21-2.11 (m; 3H); 2.02-1.88 (m; 1H); 1.60-1.44 (m; 5H); 1.36-1.13 (m; 4H); 1.08-0.93 (m; 6H); 0.93-0.67 (m; 28H).

HRMS (ESI): m/z calculated for C₇₂H₁₁₁Br₂N₁₂O₁₄ [M+H]⁺: 1525.6704;

observed: 1525.6700.

### Example 2: Synthesis of an Antibody-Drug Conjugate in Accordance with the Invention

Code for synthesized product: MF-m906-MMAE (corresponding to formula (Ia), also designated as the “antibody-drug conjugate in accordance with the invention” below or, in general, “ADC”).

Antibody used to produce the antibody-drug conjugate in accordance with the invention: m906.

Preparation of Solutions

Bioconjugation buffer: Saline buffer 1×, for example phosphate, borate, acetate, glycine, tris(hydroxymethyl)aminomethane, 4-(2-hydroxyethyl)-1-piperazineethanesulfonic acid in a pH range comprised between 6 and 9, with a final concentration of NaCl comprised between 50 and 300 mM and a final concentration of EDTA comprised between 0.1 and 10 mM.

m906 at a concentration comprised between 1 and 10 mg/mL in the bioconjugation buffer.

Reducing agent: Solution of a reducing agent selected from dithiothreitol, β-mercaptoethanol, tris(2-carboxyethyl)phosphine hydrochloride, tris(hydroxypropryl)phosphine in a concentration comprised between 0.1 and 10 mM in the bioconjugation buffer.

Linker solution: compound 8 in a concentration comprised between 0.1 and 10 mM in a mixture of organic solvents selected from dimethylsulfoxide, N,N-dimethylformamide, methanol, tetrahydrofuran, acetonitrile, N,N-dimethylacetamide, dioxane.

Bioconjugation Reaction

In an inert atmosphere, the reducing agent (4 to 100 eq) was added to m906 in the bioconjugation buffer (1 mg; 1 eq), then it was incubated in its entirety between 15° C. and 40° C. for 0.25 to 3 h, then the solution of compound 8 (4 to 100 eq) was added in an inert atmosphere and the reaction medium was stirred between 15° C. and 40° C. for 0.5 to 15 h. This reaction was duplicated, in parallel, as many times as were necessary in order to obtain the desired final quantity of ADC, i.e. 18 times.

Purification of ADC

The reaction mixture was purified over PD-10 (GE Healthcare) with PBS

Gibco buffer, pH 7.4, as many times as were necessary in order to eliminate the residual chemical reagents, i.e. purified 3 times.

Result

The steps described above enabled 12.87 mg of MF-m906-MMAE (71%) to be obtained.

### Example 3: Analyses of Antibody-Drug Coniuqate (MF-m906-MMAE)

HIC (Hydrophobic Interaction Chromatography) Analysis

Method and Apparatus

The ADC MF-m906-MMAE was diluted to 1 mg/mL with PBS, pH 7.4, before being filtered over 0.22 μm. 50 μg of the product was injected onto a MAbPac HIC-Butyl column, 5 μm, 4.6×100 mm (ThermoScientific), connected to a HPLC Waters Alliance system (e2695) equipped with a PDA (e2998) set for detection at 280 nm. The ADC MF-m906-MMAE was eluted at a rate of 1 mL/min by a gradient from 100% of buffer A (1.5 M ammonium sulfate, 50 mM monobasic sodium phosphate, 5% isopropanol (v/v), pH 7.0) to 20% of buffer B (50 mM monobasic sodium phosphate, 20% isopropanol (v/v), pH 7.0) in 2 minutes, then to 85% of buffer B in buffer A in 30 minutes, then this gradient was maintained for 1 min. The temperature was maintained at 25° C. throughout the separation.

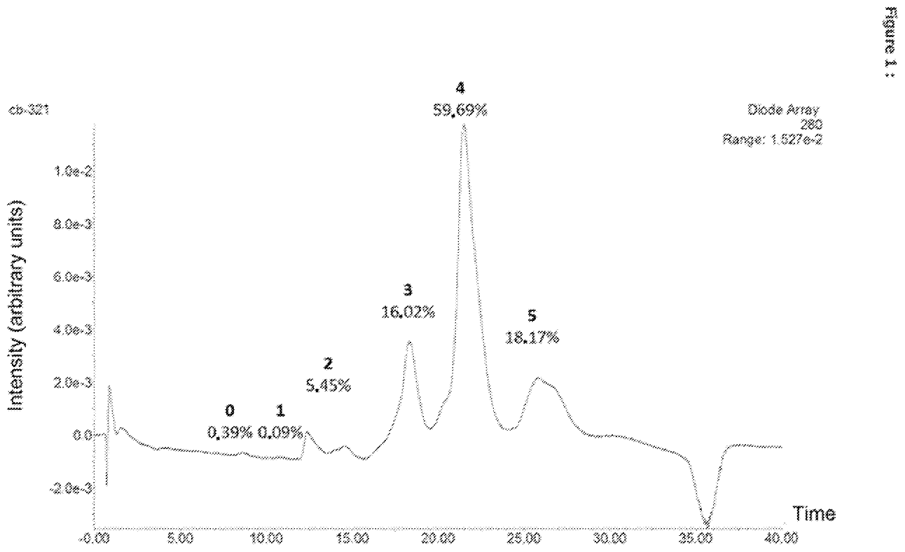

The results obtained for MF-m906-MMAE are presented in FIG. 1, and in Table 1 below.

TABLE 1

| | MF-m906-MMAE | | | | | |
|---|---|---|---|---|---|---|
| | DAR | DAR | DAR | DAR | DAR | DAR |
| | 0 | 1 | 2 | 3 | 4 | 5 |
| Retention time (min) | 8.65 | 10.87 | 12.39 | 18.36 | 21.47 | 25.9 |
| Area (%) | 0.39 | 0.09 | 5.45 | 16.20 | 59.69 | 18.17 |
| mean DAR | 3.89 | | | | | |

SEC (Size Exclusion Chromatography) Analysis

Method and Apparatus

The ADC MF-m906-MMAE was diluted to 1 mg/mL with PBS, pH 7.4, before being filtered over 0.22 μm. 50 μg of the product was injected onto an AdvanceBio SEC 2.7 μm column, 7.8×300 mm (Agilent Technologies), connected to a HPLC Waters Alliance system (e2695) equipped with a PDA (2998), set for detection at 280 nm. The ADC MF-m906-MMAE was eluted at a rate of 1 mL/min by an isocratic buffer C (1 mM monobasic sodium phosphate, 155 mM sodium chloride, 3 mM dibasic sodium phosphate, 3 mM sodium azide, pH 7.0) in 24 minutes. The temperature was maintained at 25° C. throughout the separation.

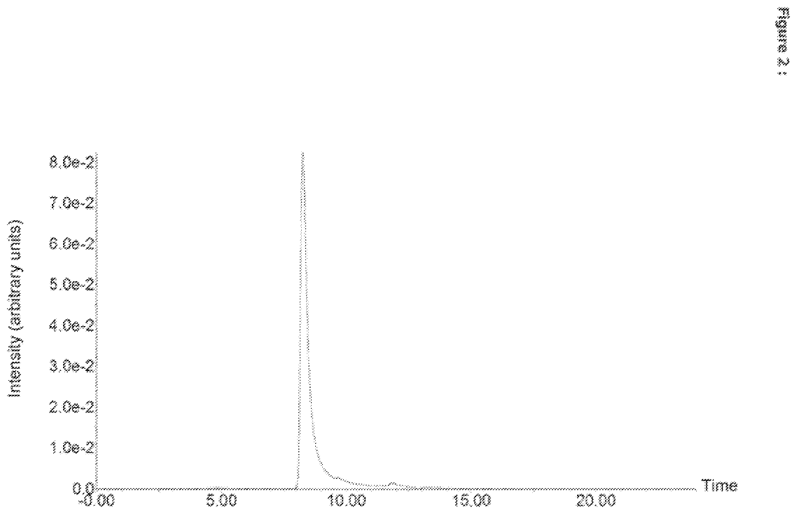

The results are presented in FIG. 2 and in Table 2 below.

TABLE 2

| MF-m906-MMAE | Aggregates | Monomers |
|---|---|---|
| Retention time (min) | 4.862 | 8.253 |
| Area (%) | 0.64 | 99.36 |

Native MS Analysis (Native Mass Spectrometry)

Method and Apparatus

The mass spectrometric analysis was carried out on a Vion IMS Qtof mass spectrometer coupled to an Acquity UPLC H-Class system from Waters (Wilmslow, UK). Before the MS analysis, the samples (20 ug) were desalinated on a BEH SEC 2.1×150 mm 300 Å desalination column by an isocratic gradient (50 mM ammonium acetate, pH 6.5) at 40 μL/min. A bypass valve was programmed to allow the solvent to enter the spectrometer between 6.5 and 9.5 min only. The MS data were acquired in positive mode with an ESI source over a m/z range of 500 to 8000 at 1 Hz and analyzed using UNIFI 1.9 software and the MaxEnt algorithm for the deconvolution.

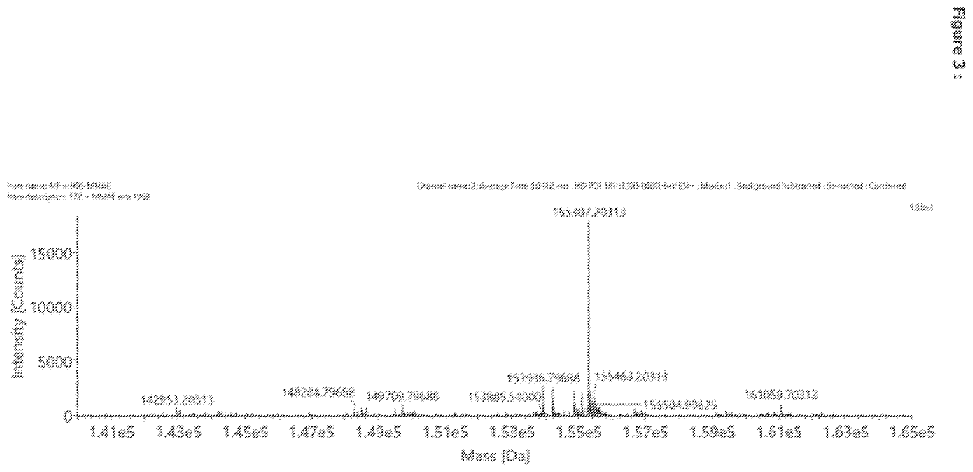

The results are presented in FIG. 3 and in Table 3 below.

TABLE 3

| MF-m906- | | | | | | |
|---|---|---|---|---|---|---|
| MMAE | DAR 0 | DAR 1 | DAR 2 | DAR 3 | DAR 4 | DAR 5 |
| MW (Da)¹ | n.o.² | n.o.² | 152572 | 153945 | 155315 | 156686 |
| Area (%) | 0 | 0 | 0.7 | 17.0 | 74.5 | 7.7 |
| mean DAR | 3.89 | | | | | |

¹G0F/G0F glycosylation
²n.o. = not observed

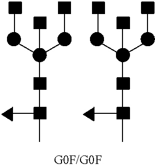

### Example 4: In Vitro Evaluation of Antibody-Drug Conjugate MF-m906-MMAE

4.1. Recognition of Tumor Cells by m906: Merkel Cell Carcinoma

Immunohistochemistry on Tumors from Patients

Method and Apparatus

Immunohistochemical labelling with a commercial anti-CD56 antibody (123C3, Ventana, Prediluted) was carried out on a cohort of 90 MCC tumors included in a micro array tissue using a benchMark XT platform and following the instructions of the supplier. The expression of CD56 was evaluated by a person skilled in the art using the following semi-quantitative score: 0: absence of expression, 1: weak and/or heterogenous positivity; 2: intense and diffuse positivity.

Results: In general, 66% of the cases were positives (n=59) with an intense and diffuse expression (score 2) detected in 37% of the cases (n=33).

Flow Cytometry and Immunohistochemistry on the MCC Lines

Method and Apparatus

In order to evaluate the expression of CD56 by the cell lines, the tumor cells were incubated in the presence of an anti-CD56 antibody coupled to FITC (BioLegend, clone: HCD56) or of the control isotype in accordance with the indications supplied by the manufacturer. The cells were then washed twice in PBS+1% fetal calf serum, then analysed.

For the evaluation of the fixation of m906 and trastuzumab (anti-HER2 antibody control) on the tumor cells, immunocytochemical labeling was carried out with the m906 antibody used at 650 ng/mL, or the trastuzumab antibody (Herceptin 150 mg Roche) at 40 ng/mL, then revealed with the aid of a secondary antibody coupled to peroxidase.

Tested Cell Lines

WaGa (RRID:CVCL_E998).

MS-1 (RRID:CVCL_E995).

PeTa (RRID:CVCL_LC73).

MKL-2 (RRID:CVCL_D027).

Results:

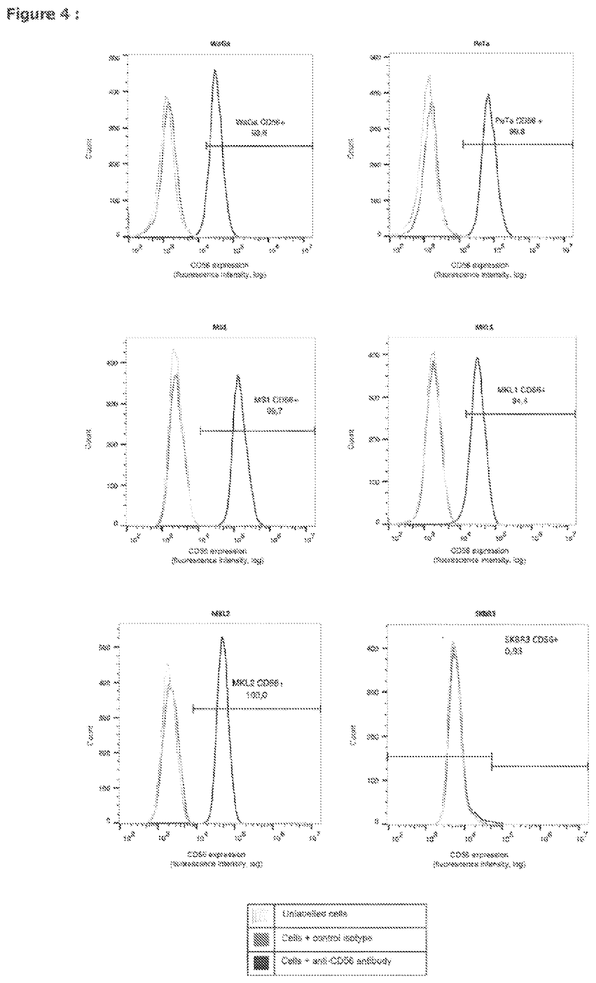

WaGa, MS-1, PeTa and MKL-2 cells are Merkel cell carcinoma cell lines (hereinafter MCC). All of the above lines are CD56-positive (FIG. 4).

SK-BR-3 (RRID:CVCL_0033): HER2-positive breast cancer line, selected as a control because it does not express CD56.

The fixations of m906 and of trastuzumab (TTZ), included as a control, were tested on the lines by immunohistochemical studies. This analysis demonstrated a fixation of m906 on all of the MCC lines, while no fixation was observed with TTZ (Table 4: confirmation of fixation of m906 on the MCC lines).

TABLE 4

| Cell line | WaGa | MS-1 | PeTa | MKL-2 | SKBR3 |
|---|---|---|---|---|---|
| Fixation of m906 | + | + | + | + | − |
| Fixation of | − | − | − | − | + |
| Trastuzumab | | | | | |

+: presence of a fixation of the antibody to the target cell revealed by peroxidase,
− absence of fixation

Imaging Flow Cytometry—m906 and Coloration of Intracellular Compartment and Image Acquisition

Method and Apparatus

In order to confirm its internalisation into the tumor cells, the antibody m906 was conjugated with Alexa Fluor 750 using the “SAIVI Rapid Antibody Labeling Kit” (Thermoscientific) following the instructions provided by the manufacturer. The WaGa cells (500 000 cells) were incubated for 30 min at ambient temperature with the conjugate m906-Alexa Fluor 750 in a buffer solution (PBS 1×, 2% fetal calf serum, 0.1% sodium azide). The cells were then fixed with the aid of BD Cytofix/Cytoperm (BD Biosciences) and permeabilized with Permwash (BD Biosciences, diluted to 1/10e) in accordance with the instructions from the manufacturer. The nuclear and lysosomal compartments were labeled with Hoechst 33342 (BD Pharmigen, 1/10000) and an anti-LAMP1 antibody coupled to phycoerythrin-Cyanine 5 (BD Pharmigen, H4A3), respectively. The analysis of the labeled cells was carried out with the aid of an Amnis® ImageStream®X Mark II flow cytometry imager (Amnis Corp., part of EMD Millipore, Seattle, Wash.) equipped with 4 lasers (375 nm, 488 nm, 642 nm and 785 nm (SSC)). The images for the WaGa cells were captured with Inspire™ imaging flow cytometry software at a magnification of 60× and with an extended depth of field (EDF).

Compensations were made before each analysis. The cells of interest were identified using the “Gradient RMS” tool of the Bright Field image (BF: white light). Debris and cellular doublets were excluded from the analysis as a function of the aspect ratio with respect to the zone of the BF image. The surface area and the mean intensity of the intracellular fluorescence (IMF) of the m906 conjugated with Alexa Fluor 750 (channel 12) was evaluated for 2 different incubation times (5 and 30 min) with the aid of the “surface mask” and “cytoplasm mask” tools, using the IDEAS® v6.2 software. The internalisation score for the m906 conjugated with Alexa Fluor 750 (channel 12) was determined using the internalisation function, allowing the ratio of the intensity of intracellular fluorescence to the intensity of the whole cells to be defined. Cells with internalized antibodies have positive scores. The co-localization assistant using the “Bright Detail Similarity” (BDS) function was employed in order to quantify the level of co-localization between the anti-LAMP1 antibody conjugated with phycoerythrin-Cyanine 5 (channel 5) and the m906 conjugated with Alexa Fluor 750 (channel 12). The positive score for BDS (n. 1) indicates lysosomal localization for the m906.

Results

The m906 anti-CD56 antibody is internalized in the lysosome in WaGa cells expressing CD56 (Table 5). This is a crucial step for the release of drugs by an ADC, which makes m906 a good candidate antibody for the development of an ADC.

TABLE 5

| | | m906 | Intracellular | Internal- | BDS*² |
|---|---|---|---|---|---|
| Incubation | Number | fluorescence | fluorescence | isation | score |
| period | of cells | surface | of m906 | score for | (LAMP1/ |
| (minutes) | in focus | (MFI*¹) | (MFI*¹) | m906 | m906) |
| 5 | 8617 | 930 | 50046 | 7.08 | 1.4 |
| 30 | 5223 | 580 | 50464 | 7.96 | 1.4 |

*¹Mean Fluorescence Intensity
*²Bright Detail Similarity

4.2. Cytotoxicity of MF-m906-MMAE on Lines In Vitro

Evaluation of Viability (Proliferation Test)

Method and Apparatus

In order to evaluate cell viability; a cytotoxicity test using XTT was carried out. The cells were deposited in a 96-well plate (50 000 cells/well) in 7 replicates. The ADCs MF-m906-MMAE and MF-TTZ-MMAE (ADC control) were added in incremental concentrations. The culture medium acted as a negative control. After 4 days of exposure to the drug, 25 μL of reagent XTT was added per well and the absorption was measured at 450 nm after 4 hours of incubation at 37° C. The absorption at 620 nm was used as a reference.

Results

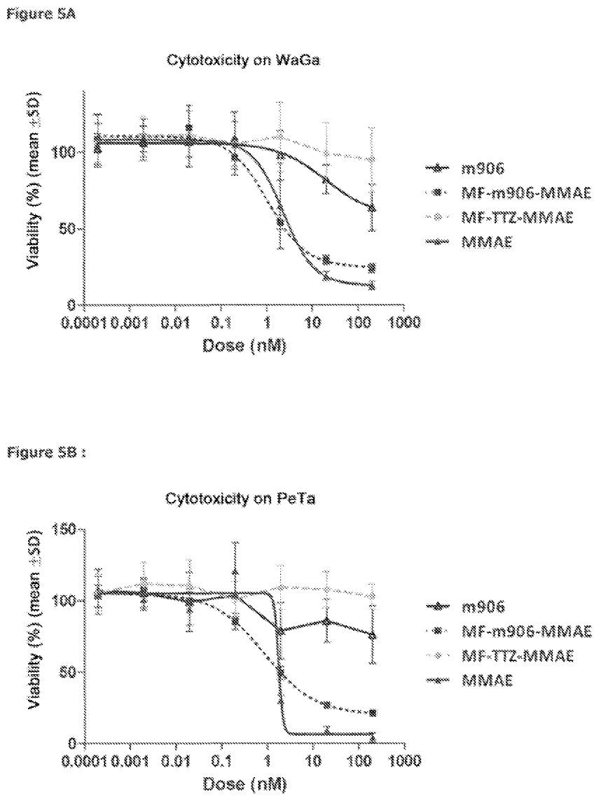

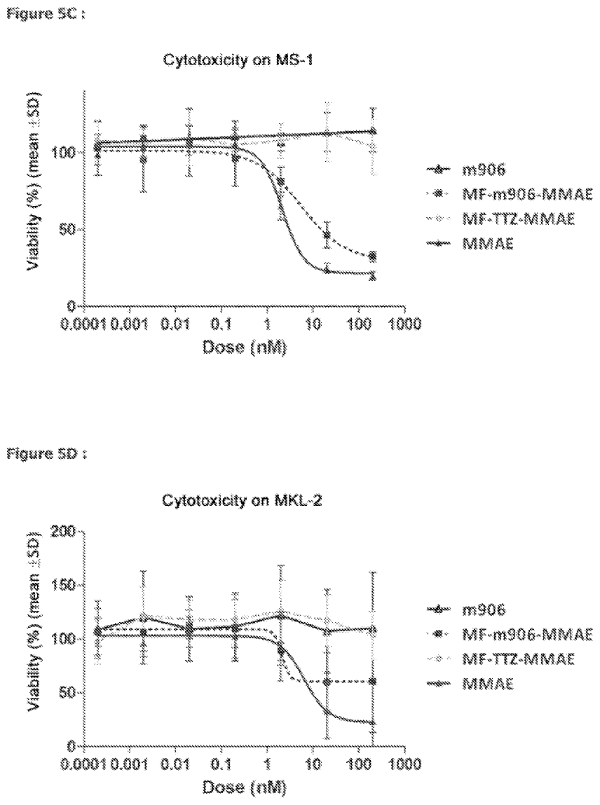

The evaluation of the cytotoxicity on cell lines has shown that the ADC MF-m906-MMAE is as cytotoxic as free MMAE (IC₅₀ between 1-10 nM) for all of the MCC lines. Furthermore, neither the antibody m906 (i.e. non coupled to MMAE), nor the ADC MF-TTZ-MMAE (ADC control) has a cytotoxic effect on these same lines at the lowest effective concentrations tested, demonstrated the absence of intrinsic toxicity of the construct (FIG. 5).

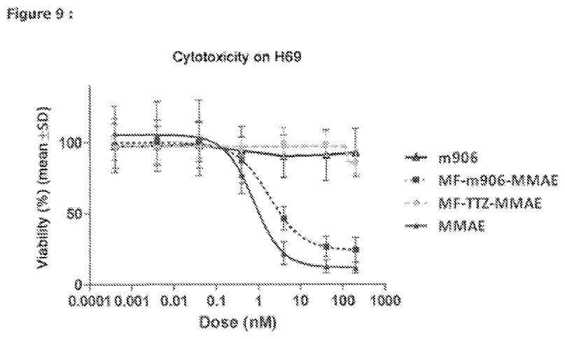

The evaluation of cytotoxicity on the H69 line (RRID:CVCL_1579), a small cell lung carcinoma cell line, has shown that the ADC MF-m906-MMAE is as cytotoxic as free MMAE (IC₅₀ between 1-2 nM). Furthermore, neither the antibody m906 (i.e. non coupled to MMAE), nor the ADC MF-TTZ-MMAE (ADC control) has a cytotoxic effect on this line at the lowest effective concentrations tested, demonstrating the absence of intrinsic toxicity of the construct (FIG. 9).

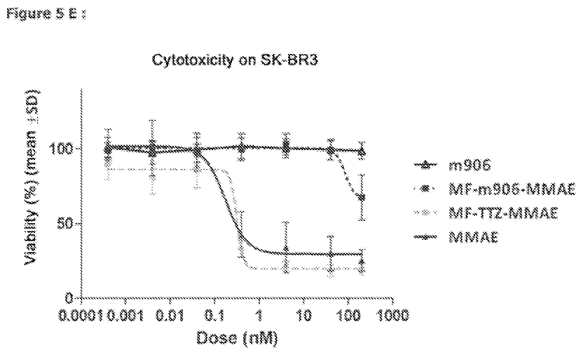

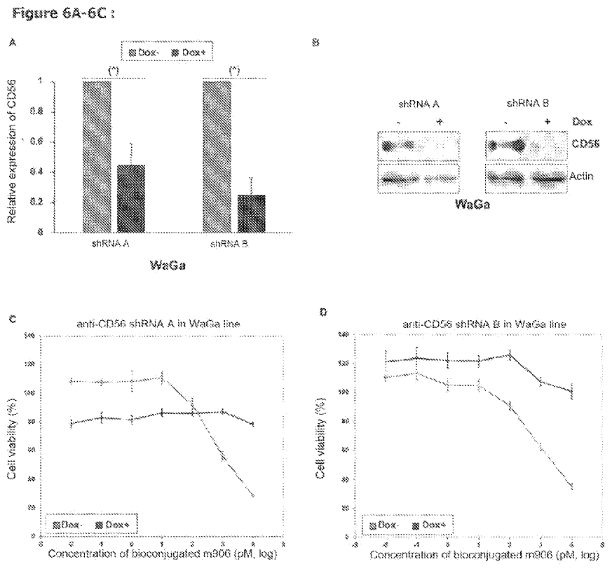

4.3. Confirmation of the Specificity of m906: The Cytotoxic Effect Observed is Dependent on CD56 (FIG. 6)

Plasmids and Lentiviral Transduction

Method and Apparatus

Three shRNAs targeting the sequence for CD56 were generated (sequences obtained from Consortium RNAi (A: TRCN0000373085 (SEQ ID NO 9-10)/B: TRCN0000373034 (SEQ ID NO.11-12)/C: TRCN0000073460 (SEQ ID NO.13-14)) and cloned in a FH1tUTG lentiviral vector, as described above [5]. Note in this construct that the activity of the promoter controlling the transcription of sequences of shRNA can be induced by doxycycline. The lentiviral supernatants were produced in the HEK293T (RRID:CVCL_0063) cells as described above [6-7]. The harvested supernatant was sterilized by filtration (0.45 μm) and polybrene was added (1 μg/mL) before infection. After 14-20 h of incubation with the supernatants containing the lentiviruses, the target cells were washed then underwent antibiotic selection (puromycin). For invalidation of the expression of CD56 in the tumor lines (knock-down), the cells were exposed to doxycycline for 7 days before analysis.

Results

The cytotoxicity induced by MF-m906-MMAE was substantially reduced during CD56 knock-down and this was the case for the three shRNAs (FIG. 6), confirming that the recognition of CD56 by m906 is essential in order to induce a cytotoxicity for MF-m906-MMAE.

### Example 5: In Vivo Evaluation of Antibody-Drug Conjugate (MF-m906-MMAE)

Therapeutic Performance of m906 in a Xenograft Model of MCC Cell Lines on

NOD/SCID mouse

Method and Apparatus

Mouse Model

Twenty 7-week-old female NOD/SCID mice (Janvier Labs) were kept under aseptic conditions. All of the procedures relating to the animals were approved by the local ethics committee (Apafis-10076-2017053015488124 v4). The CD56-positive MCC cell line “WaGa” [8] was used for tumor induction. The mice, anesthetized with isoflurane, received a subcutaneous injection of 10⁷ cells in Matrigel (injection site: back). The tumor size, determined by measuring the width, the length and the height with a calliper and the general condition of the animals were monitored every 2 days throughout the procedure. The tumor volume was determined using the following formula: width×length×height×π/6. When the volume of the tumor reached 50 mm³, the mice were included in the study and randomly assigned to the experimental or control groups.

Experimental Procedure

After inclusion, the animals received an intravenous injection of ADC (either MF-m906-MMAE or MF-TTZ-MMAE, 10 mg/kg) or the injection of an equivalent volume of PBS for the control mice. A new injection was made in the event of doubling of the volume of the tumor in the experimental group. The mice were sacrificed 30 days after inclusion or if a critical point was reached (tumor ulceration, loss of 20% of weight or prostration). An autopsy was carried out on the animals and all of the organs were examined macroscopically by a pathologist. The weight and volume of the tumors were evaluated after dissection. Microscopic examination of the tumors, the lungs and the liver was carried out in order to detect any potential metastatic progression.

Statistical Analysis

Continuous data were described using means and limits. Categorical data were described by numbers and percentages of interpretable cases. The combinations were evaluated using Fisher's exact test for the categorical data or by Mann-Whitney or Kruskall Wallis tests for the continuous data. The statistical analyses were carried out using XLStat software (Addinsoft, Paris, France). p<0.05 was considered to be statistically significant.

Results

MF-m906-MMAE reduced tumor growth in a murine xenograft model of MCC cell lines.

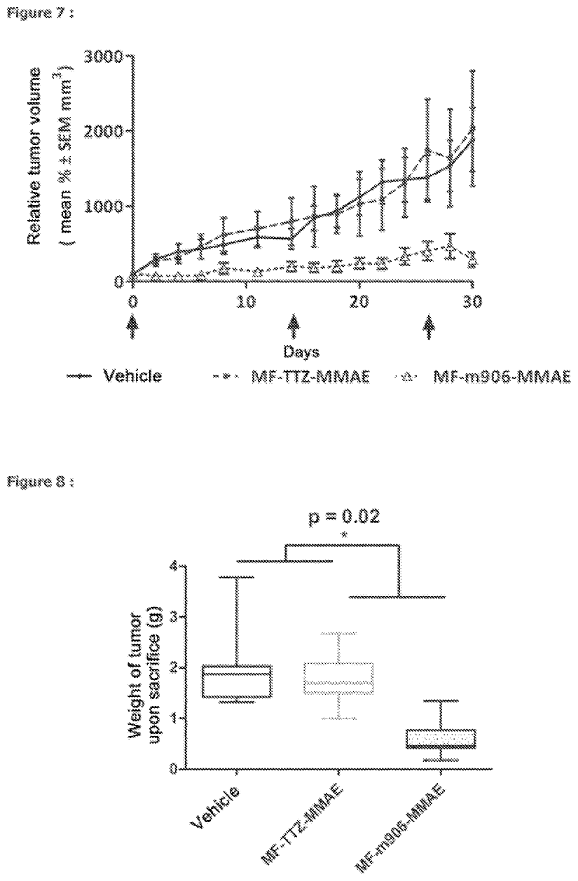

The results of the administration of a triple dose of 10 mg/kg of MF-m906-MMAE, MF-TTZ-MMAE or PBS are presented in FIG. 7 (relative tumor volumes) and FIG. 8 (tumor mass at end of study). In the last two groups, used as controls, a similar tumor growth was observed with a mean coefficient for the slope of the growth curve of 105% of the initial tumor volume per day (limits 35-166) and 108% of the initial tumor volume per day (limits 33-318) for the PBS and MF-TTZ-MMAE groups respectively). For the experimental group treated with MF-m906-MMAE, tumor growth was significantly retarded (mean coefficient for the slope of the growth curve of 18% of the initial tumor volume per day (limits 1-53); Kruskal Wallis test: p=0.01). As a consequence, the final median weight of the tumors was reduced in the group treated with MF-m906-MMAE compared with the control animals (FIG. 8) (mean weight 0.7 g (limits: 0.4-1.3) as opposed to 2.03 g (limits: 1.3-3.7) and 1.78 g (limits: 1-2.7), Kruskal Wallis test: p-0.02). After sacrifice, no metastasis was observed either in the experimental group or in the control groups. No signs of MF-m906-MMAE toxicity were observed on the non-tumoral tissues removed during autopsy.

### SEQUENCE LISTING

TABLE 6

| Sequence number | Sequence type | Amino acid sequence |
|---|---|---|
| SEQ ID NO: 1 | CDR1 of the light | QSLLHSNGYN |
| | chain of m906 | |
| SEQ ID NO: 2 | CDR2 of the light | YLG |
| | chain of m906 | |
| SEQ ID NO: 3 | CDR3 of the light | CMQSLQTPWT |
| | chain of m906 | |
| SEQ ID NO: 4 | CDR1 of the heavy | GGTFTGYYMHW |
| | chain of m906 | |
| SEQ ID NO: 5 | CDR2 of the heavy | NSGGTNYAQ |
| | chain of m906 | |
| SEQ ID NO: 6 | CDR3 of the heavy | LSSGYSGYFDYWGQG |
| | chain of m906 | |
| SEQ ID NO: 7 | Light chain of m906 | DVVMTQSPLSLPVTPGEPASIS |
| | | CRSSQSLLHSNGYNFLDWYLQ |
| | | KPGQSPQLLIYLGSNRASGVP |
| | | DRFSGSGSGTDFTLKISRVEA |
| | | DDVGVYYCMQSLQTPWTFGH |
| | | GTKVEIKRTVAAPSVFIFPPSD |
| | | EQLKSGTASVVCLLNNFYPRE |
| | | AKVQWKVDNALQSGNSQESV |
| | | TEQDSKDSTYSLSSTLTLSKAD |
| | | YEKHKVYACEVTHQGLSSPVT |
| | | KSFNRGEC |
| SEQ ID NO: 8 | Heavy chain of | EVQLVQSGAEVKKPGSSVKVS |
| | m906 | CKASGGTFTGYYMHWVRQAP |
| | | GQGLEWMGWINPNSGGTNYA |
| | | QKFQGRVTMTRDTSISTAYME |
| | | LSRLRSDDTAVYYCARDLSSG |
| | | YSGYFDYWGQGTLVTVSSAST |
| | | KGPSVFPLAPSSKSTSGGTAA |
| | | LGCLVKDYFPEPVTVSWNSGA |
| | | LTSGVHTFPAVLQSSGLYSLSS |
| | | VVTVPSSSLGTQTYICNVNHKP |
| | | SNTKVDKKVEPKSCDKTHTCP |
| | | PCPAPELLGGPSVFLFPPKPK |
| | | DTLMISRTPEVTCVVVDVSHE |
| | | DPEVKFNWYVDGVEVHNAKT |
| | | KPREEQYNSTYRVVSVLTVLH |
| | | QDWLNGKEYKCKVSNKALPA |
| | | PIEKTISKAKGQPREPQVYTLP |
| | | PSRDELTKNQVSLTCLVKGFY |
| | | PSDIAVEWESNGQPENNYKTT |
| | | PPVLDSDGSFFLYSKLTVDKS |
| | | RWQQGNVFSCSVMHEALHNH |
| | | YTQKSLSLSPGK |
| SEQ ID NO: 9 | RNAi | TCCCAGCGTTGGAGAGTCCA |
| | TRCN0000373085 | AATTCTCGAGAATTTGGACTC |
| | Forward | TCCAACGCTTTTTTCGGG |
| SEQ ID NO: 10 | RNAi | AAAAAAGCGTTGGAGAGTCC |
| | TRCN0000373085 | AAATTCTCGAGAATTTGGACT |
| | Reverse | CTCCAACGCT |
| SEQ ID NO: 11 | RNAi | TCCCCGTTCCCTGAAACCGT |
| | TRCN0000373034 | TAAACTCGAGTTTAACGGTTT |
| | Forward | CAGGGAACGTTTTT |
| SEQ ID NO: 12 | RNAi | CGGGAAAAACGTTCCCTGAA |
| | TRCN0000373034 | ACCGTTAAACTCGAGTTTAAC |
| | Reverse | GGTTTCAGGGAACG |
| SEQ ID NO: 13 | RNAi | TCCCCATGTACCTTGAAGTG |
| | TRCN0000073460 | CAATCTCGAGATTGCACTTCA |
| | Forward | AGGTACATGTTTTT |
| SEQ ID NO: 14 | RNAi | CGGGAAAAACATGTACCTTG |
| | TRCN0000073460 | AAGTGCAATCTCGAGATTGC |
| | Reverse | ACTTCAAGGTACATG |
| SEQ ID NO: 15 | Variable domain of | DVVMTQSPLSLPVTPGEPASIS |
| | the light chain of | CRSSQSLLHSNGYNFLDWYLQ |
| | m906 | KPGQSPQLLIYLGSNRASGVP |
| | | DRFSGSGSGTDFTLKISRVEA |
| | | DDVGVYYCMQSLQTPWTFGH |
| | | GTKVEIKR |
| SEQ ID NO: 16 | Variable domain of | EVQLVQSGAEVKKPGSSVKVS |
| | the heavy chain of | CKASGGTFTGYYMHWVRQAP |
| | m906 | GQGLEWMGWINPNSGGTNYA |
| | | QKFQGRVTMTRDTSISTAYME |
| | | LSRLRSDDTAVYYCARDLSSG |
| | | YSGYFDYWGQGTLVTVS |

### REFERENCES CITED IN THE FORMAT “[REFERENCE NUMBER]”

1. Feng, Y. et al., MAbs, May-June; 8(4): 799-810, 2016
2. Goyon, A et al., J. Chromatogr. B, 1065-1066, 35-43, 2017
3. Fekete, S. et al., J. Pharm. Biomed. Anal., 130, 3-18, 2016
4. Barran, P. et al., EuPA Open Proteomics, 11, 23-27, 2016
5. Houben, R. et al., International journal of Cancer, 130, 847-856, 2012
6. Angermeyer S. and al., J. Invest. Dermatol., 133(8):2059-2064.2013
7. Aragaki M. et al., Biochem. Biophys. Res. Commun., 368(4):923-929, 2008
8. Houben R, et al., J. Virol.; 84(14):7064-7072, 2010

1. A conjugate antibody-drug with the following formula (I): 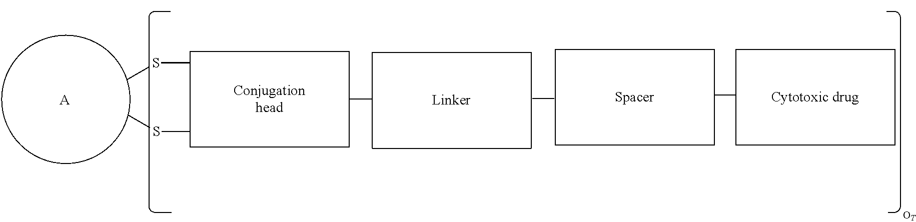
in which:
A is an anti-CD56 antibody or an antibody fragment;
the attachment head is represented by one of the following formulae: 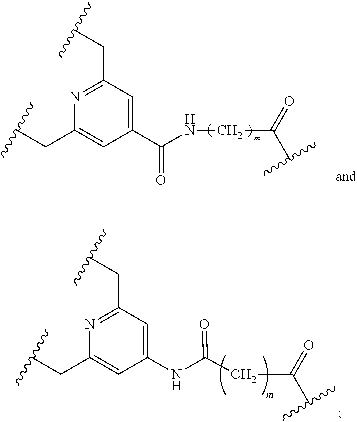
the linker is a cleavable linker represented by the following formula: 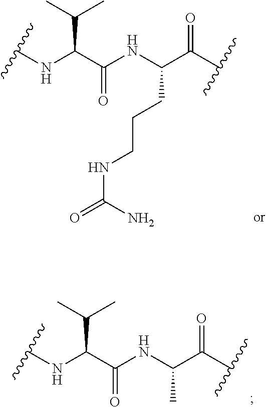 [SMILES: *N[C@H](C(=O)N[C@@H](CCCNC(N)=O)C(*)=O)C(C)C ; *N[C@H](C(=O)N[C@@H](C)C(*)=O)C(C)C]
the spacer is represented by the following formula: 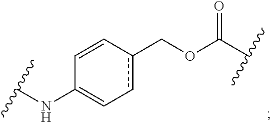
m is an integer from 1 to 10;
n is an integer from 1 to 4.

2. The conjugate antibody-drug as claimed in claim 1, in which m is equal to 4 or 5.

3. The conjugate antibody-drug as claimed in claim 1, in which the cytotoxic drug is selected from methotrexate, IMIDs, duocarmycin, combretastatin, calicheamicin, monomethyl auristatin E (MMAE), monomethyl auristatin F (MMAF), maytansine, DM1, DM4, SN38, amanitin, pyrrolobenzodiazepine, pyrrolobenzodiazepine dimer, pyrrolopyridodiazepine, pyrrolopyridodiazepine dimer, an inhibitor of histone deacetylase, an inhibitor of tyrosine kinase, and ricin, preferably MMAE.

4. The conjugate antibody-drug as claimed in claim 1, with the following formula:

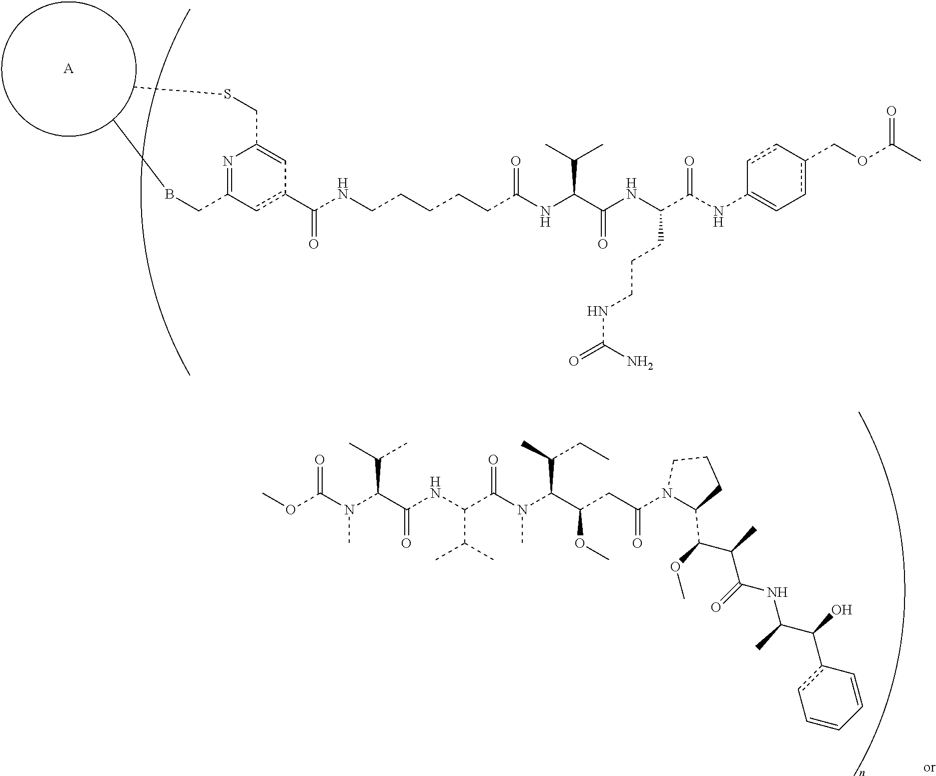

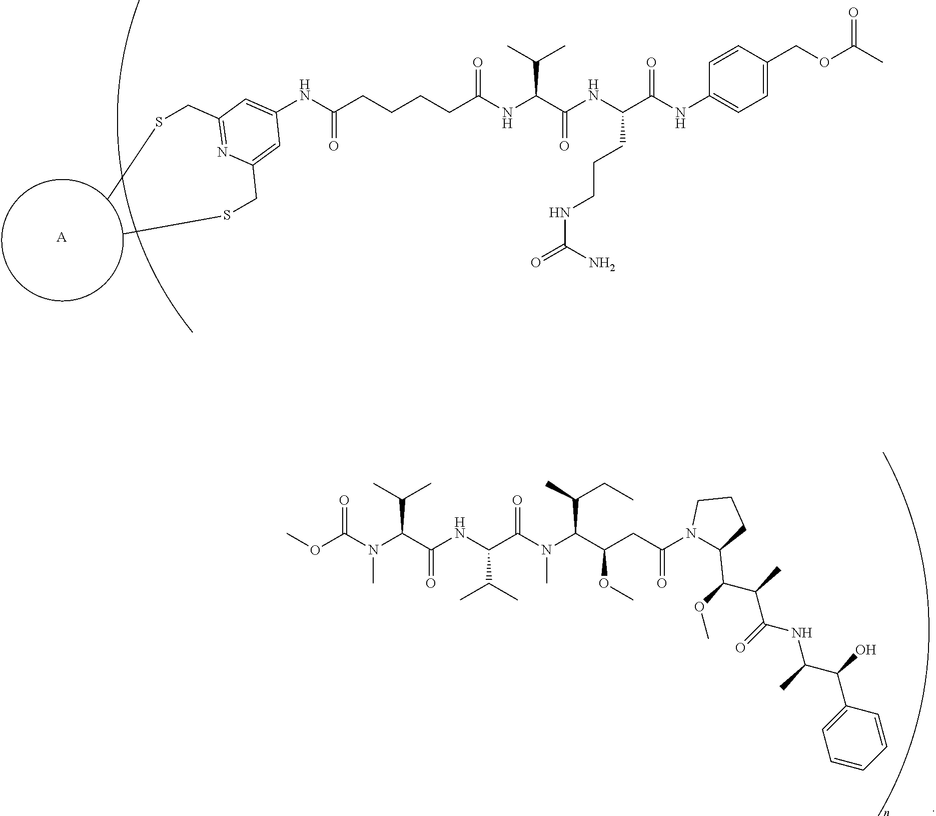

5. The conjugate antibody-drug as claimed in claim 1, in which A is m906.

6. The conjugate antibody-drug as claimed in claim 1, with the following formula (Ia): 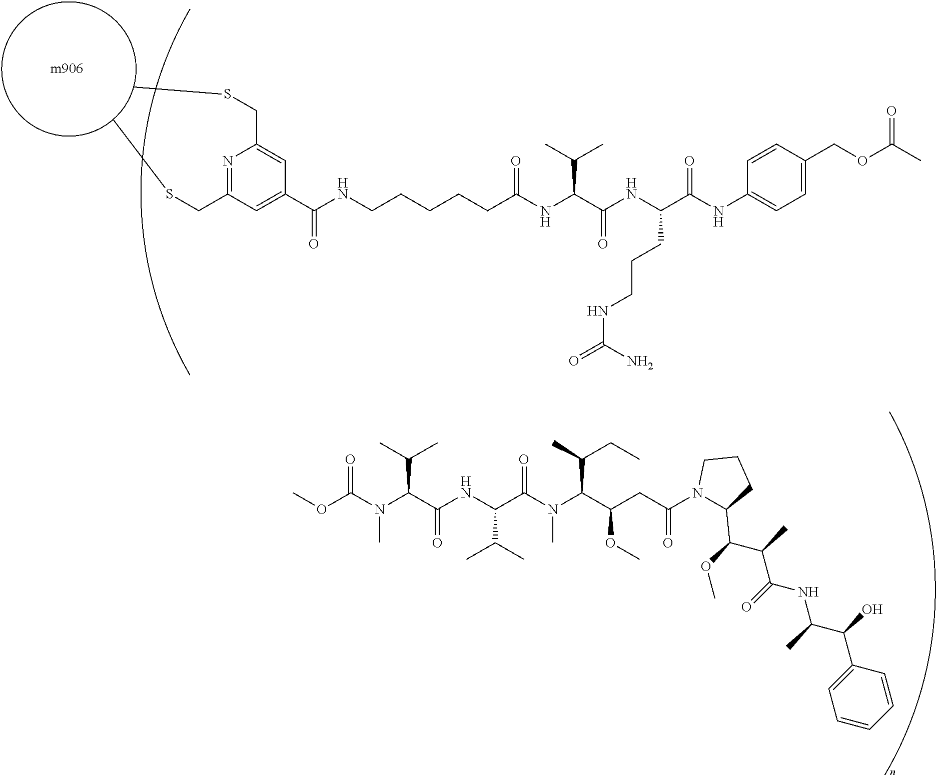
or with the following formula (Ia′):

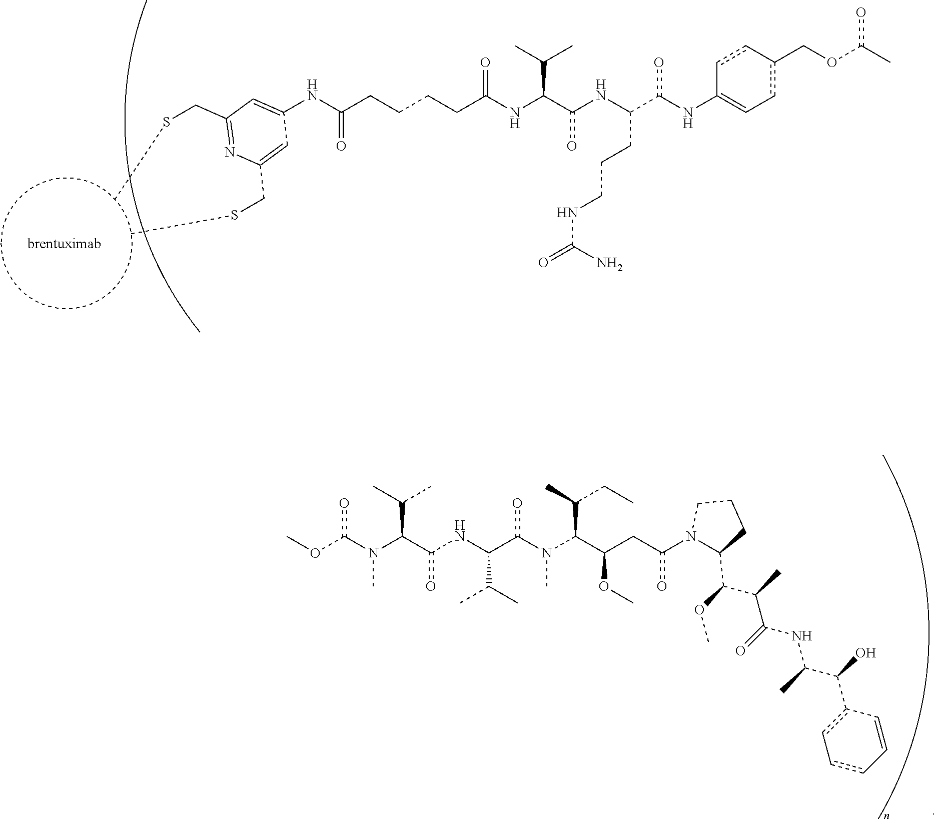

7. A composition comprising one or more antibody-drug conjugate(s) as claimed in claim 1.

8. The composition as claimed in claim 7, in which at least 50%, preferably approximately 60%, of the antibody-drug conjugates have an n equal to 4.

9. The composition as claimed in claim 7, in which A is an antibody and the mean Drug-to-Antibody Ratio (mean DAR) is comprised between 3.5 and 4, preferably comprised between 3.8 and 4, for example equal to 3.9±0.1.

10. The composition as claimed in claim 7, further comprising paclitaxel, docetaxel, doxorubicin and/or cyclophosphamide, lenalidomide, dexamethasone, carboplatin, etoposide and/or an antibody used in anti-cancer immunotherapy such as an anti-PD1 or an anti-PD-L1.

11. The conjugate antibody-drug as claimed in claim 1, for use as a drug.

12. The conjugate antibody-drug as claimed in claim 1, for use in the treatment of a CD56+ cancer, preferably melanoma, blastemal tumors, hemopathies such as acute myeloid leukemias, myelomas, blastic plasmacytoid dendritic cell neoplasms and neuroendocrinal carcinomas.

13. The conjugate antibody-drug for use as claimed in claim 12, in which the CD56+ cancer is selected from neuroendocrinal carcinomas such as small cell lung carcinoma or Merkel cell carcinoma, preferably Merkel cell carcinoma.

14. A method for the preparation of an antibody-drug conjugate as claimed in claim 1, comprising the following steps:
(i) preparing a cytotoxic conjugate by coupling a attachment head with formula: 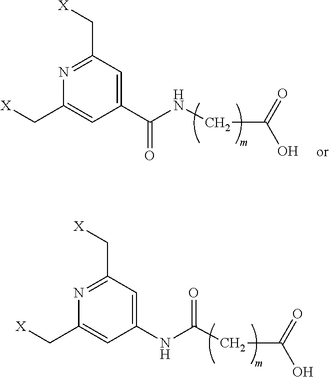
to a compound with formula: 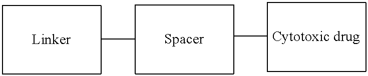
in which:
the linker is a cleavable linker selected from the following formulae: 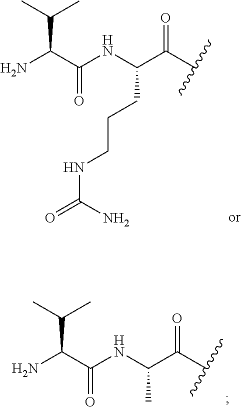 [SMILES: *C(=O)[C@H](CCCNC(N)=O)NC(=O)[C@@H](N)C(C)C ; *C(=O)[C@H](C)NC(=O)[C@@H](N)C(C)C]
the spacer is represented by the following formula: 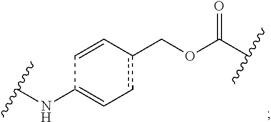
X is Br, Cl, I or F;
m is an integer from 1 to 10, advantageously from 2 to 7, from 3 to 6, advantageously equal to 4 or 5; and
(ii) reacting the cytotoxic conjugate obtained in step (i) with an anti-CD56 antibody or an anti-CD56 antibody fragment.

15. The method for the preparation of an antibody-drug conjugate as claimed in claim 14, comprising a step that consists of reacting MMAE 6-(2,6-bis(bromomethyl)pyridin-4-yl)amido-N-hexanamide-valine-citrulline-p-aminobenzoyl carbamate or MMAE 6-((2,6-bis(bromomethyl)pyridin-4-yl)amino)-6-oxohexanamide-valine-citrulline-p-aminobenzoyl carbamate with an anti-CD56 antibody or an anti-CD56 antibody fragment.

16. The composition as claimed in claim 7, for use as a drug.

17. The composition as claimed in claim 10, for use in the treatment of a CD56+ cancer, preferably melanoma, blastemal tumors, hemopathies such as acute myeloid leukemias, myelomas, blastic plasmacytoid dendritic cell neoplasms and neuroendocrinal carcinomas.
